(19) United States
(12) Patent Application Publication
(10) Pub. No.: US 2023/0250467 A1
(43) Pub. Date: Aug. 10, 2023

(54) OFF-TARGET BLOCKING SEQUENCES TO IMPROVE TARGET DISCRIMINATION BY POLYMERASE CHAIN REACTION

(71) Applicant: GT Molecular, Inc., Fort Collins, CO (US)

(72) Inventors: Sarah KANE, Fort Collins, CO (US); Rose NASH, Fort Collins, CO (US); Karl RAVET, Fort Collins, CO (US); Stephanie BARBARI, Fort Collins, CO (US)

(21) Appl. No.: 18/049,563

(22) Filed: Oct. 25, 2022

Related U.S. Application Data
(60) Provisional application No. 63/271,522, filed on Oct. 25, 2021.

(51) Int. Cl. C12Q 1/6809; C12Q 1/6827; C12Q 1/686; C12Q 1/6886

(52) U.S. Cl. CPC C12Q 1/6809; C12Q 1/6827; C12Q 1/686; C12Q 1/6886; C12Q 2600/156

(57) ABSTRACT

Provided herein are methods and kits for discriminating a target sequence from a reference sequence in a polymerase chain reaction (PCR) by use of a promiscuity-blocking nucleotide juror (PBNJ) oligonucleotide.

### CROSS REFERENCE TO RELATED APPLICATIONS

This patent application claims the benefit of U.S. Provisional Patent Application No. 63/271,522, filed Oct. 25, 2021, which is incorporated by reference herein to the extent not inconsistent herewith.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One XML file named “97-21US_Seq_Listing_12-5-2022.XML”, created with WIPO Sequence on Dec. 5, 2022 of file size 72,538 KB.

### FIELD OF THE INVENTION

The present application relates generally to assays for the detection and quantification of PCR products using oligonucleotides to reduce or avoid detection of off-target amplification products.

### BACKGROUND

The following description provides a summary of information relevant to the present application and is not an admission that any of the information provided or publications referenced herein is prior art to the present application.

PCR is a method of making copies of DNA samples, wherein new strands of DNA complementary to a template DNA or RNA strand(s) are generated by using primers to the template strands and DNA polymerase for extension of the primers. Quantitative PCR (qPCR, also referred to as real-time PCR) is a PCR-based technique that couples amplification of a target DNA sequence with quantification of the concentration of DNA in the reaction. Digital PCR (dPCR) is a refinement of conventional PCR methods, which applies partitioning or droplet formation of samples into subsamples, and can be used to directly quantify and clonally amplify nucleic acids strands. Reverse transcription PCR (RT-PCR) combines reverse transcription of RNA into complementary DNA and PCR, wherein the PCR can be either dPCR or qPCR. In dPCR or qPCR, labeled probes designed to hybridize specifically to DNA target regions are used to detect and quantify amplified DNA. Yet, probes may hybridize non-specifically to non-target regions which can substantially reduce the accuracy of DNA/RNA detection and quantification. Non-Specific hybridization to a non-target region is referred to as promiscuity. In addition, some off-target amplification is expected which may also impact accuracy. Provided herein are methods, kits, and related compositions that address the need in the art for methods and kits to accurately detect and quantify DNA in dPCR or in qPCR or RNA by RT-PCR (e.g. RT-dPCR and RT-qPCR), including for very small levels of a desired target nucleic acid.

### SUMMARY

The accuracy of dPCR and qPCR assays (for DNA or complementary DNA reverse transcribed from RNA) is limited by non-specific binding of labeled probes and/or off-target amplification, leading to inaccuracy in detection and quantification of a target sequence. The present disclosure provides methods and kits for discriminating a target sequence from a reference sequence in a biological sample by PCR, including dPCR or qPCR, by reduction of non-specific binding of labeled probes. The methods, kits and compositions provided herein are useful for discrimination of a biological material from any of a range of organisms, wherein the target sequence originates from a natural variation or from an artificial modification, such as mutagenesis or genome editing. The difference between target and reference may be as small as a 1 base pair (bp) substitution, or may correspond to indels (insertion or deletion). Irrespective of the type of nucleic acid difference, provided herein is a platform that increases dPCR and/or qPCR accuracy to reliably detect any of a range of differences in sequence between target and reference sequences that otherwise is not reliably detectable due to off-target binding and associated amplification and detection. By reducing detection of non-target sequences, the on-target amplification is more reliably detected, even at very low concentrations relative to reference sequences. The methods, kits and compositions are particularly useful as they can be incorporated into conventional “probe-based” PCR assays, thereby providing high-efficiency and accurate detection without a need for additional complex, time-consuming or expensive components.

The methods, kits and compositions are compatible with a range of targets used in PCR-based detection and diagnosis, wherein an at least one nucleotide difference is desirably detected and so suppression of off target amplification is important so as to better discriminate the target sequence from the reference sequence.

Provided herein are various methods, described in this section as “aspects”, such as Aspect 1: A method of discriminating a target sequence from a reference sequence in a biological sample by polymerase chain reaction (PCR), the method comprising the steps of:
a. providing a labeled probe comprising a fluorophore and a quencher that is complementarity to and specifically binds to the target sequence and may non-specifically bind to the reference sequence;
b. providing a promiscuity-blocking nucleotide juror oligonucleotide (PBNJ) that specifically binds to the reference sequence and may non-specifically bind to the target sequence, wherein the PBNJ comprises a reference binding region and an extension blocker that prevents elongation by a polymerase;
c. performing PCR on a solution comprising:
   i. a sample containing the reference and/or target sequence;
   ii. the labeled probe;
   iii. the PBNJ at a competitive concentration relative to a concentration of the labeled probe;
   iv. PCR reagents; and
      wherein the PBNJ specific binding to the reference sequence is competitive to the labeled probe binding to the reference sequence and suppresses labeled probe bound to the reference sequence relative to amplification; thereby discriminating the target sequence from the reference sequence. The competitive concentration of PBNJ:probe will depend on the application of interest and, therefore, is compatible with a range of concentrations. Examples include, but are not limited to ratios of between 0.2 and 20.

2. The method of aspect 1, wherein the biological sample is selected from the group consisting of:
a. viruses, wherein the reference sequence is from a wild-type virus or a parent virus and the target sequence comprises at least one mutation in the reference sequence;
b. mammalian cells, wherein the reference sequence is reflective of a low-disease condition state and the target sequence has one or more nucleotide changes in the reference sequence reflective of an elevated disease condition risk or the presence of disease;
c. circulating cell free tumor DNA, wherein the reference sequence is somatic, wild-type sequence and the target sequence originated in a tumor or cancerous cell and has one or more nucleotide changes in the reference sequence reflective of an elevated disease condition risk or the presence of disease;
d. circulating cell free fetal DNA, wherein the reference sequence is reflective of the maternal DNA sequence and the target sequence has one or more nucleotide changes in the reference sequence reflective of the fetus DNA sequence;
e. bacteria, wherein the reference sequence is from a wild-type bacterium or one species of bacteria and the target sequence comprises at least one variation in the reference sequence;
f. fungus, wherein the reference sequence is from a wild-type fungus or one species of fungus and the target sequence comprises at least one variation in the reference sequence;
g. plants, wherein the reference sequence is from a wild-type plant or one species of plant and the target sequence comprises at least one variation in the reference sequence; and

Of course, the invention is also compatible with sequences relevant for other applications, such as ‘genetic testing’ (for different diseases such as Alzheimer's disease, cancer, cystic fibrosis, sickle cell anemia, Duchenne muscular dystrophy, thalassemia, Huntington's disease, rare diseases, and other diseases), and a range of applications (cancer diagnosis, genetic disease diagnosis, cardiovascular disease diagnosis, and others). Another important application is for detection of antimicrobial resistance (AMR) genes, where small changes in the microbe genes can lead to antimicrobial resistance. Prompt and reliable early detection of such AMR is important for health outcomes and improved safety, including in the food, beverage, agricultural (including cannabis) industries, particularly for E. coli, Salmonella, and any of a variety of bacterial targets identified by the CDC as of concern.

3. The method of aspect 1 or 2, wherein the PBNJ eliminates ≥90% detection of non-specific amplification of the reference sequence. Of course, the methods and kits provided herein are compatible with less than 90% elimination, based on obtaining a meaningful threshold.

4. The method of any one of aspects 1 to 3, wherein the reference and target sequences differ by:
a. a single nucleotide substitution;
b. a nucleotide insertion of one or more nucleotides; and/or
c. a nucleotide deletion of one or more nucleotides.

5. The method of any one of aspects 1 to 4, wherein the reference and target sequences are DNA sequences or RNA sequences.

6. The method of any one of aspects 1 to 5, wherein the PCR is selected from the group consisting of dPCR, qPCR, RT-dPCR and RT-qPCR.

7. The method of any one of aspects 1 to 6, wherein the labeled probe is a dual-label probe comprising a fluorescent molecule and a quencher molecule.

8. The method of any one of aspects 1 to 7, wherein the labeled probe is a single-nucleotide variant (SNV)-specific TaqMan® probe (a fluorophore covalently attached to a 5′ end of the probe and a quencher at a 3′ end of the probe or an internal quencher).

9. The method of any one of aspects 1 to 8, for detection of a SNV, insertion or deletion containing DNA or RNA sequences.

10. The method of any of aspects 1 to 9, wherein the PCR is dPCR and the dPCR comprises partition or droplet-based PCR and the PBNJ reduces or eliminates signal associated with a lower efficiency, non-specific off-target amplification, thereby increasing a signal to noise ratio for specific amplification of the target sequence.

11. The method of any one of aspects 1 to 10, further comprising the steps of:
a. tuning a probe output amplitude by providing the PBNJ at a lower concentration; and
b. detecting a plurality of probe output amplitudes for multiplex detection of a plurality of target sequences in a single or a multichannel fluorescence detector.

12. The method of any one of aspects 1 to 11, wherein the target and reference sequence differ by a single nucleotide mismatch that is a single nucleotide polymorphism (SNP) or is part of a short nucleotide polymorphism.

13. The method of any one of aspects 1 to 11, wherein the target and reference sequence differ by an insertion.

14. The method of any one of aspects 1 to 11, wherein the target and reference sequence differ by a deletion.

15. The method of any one of aspects 1 to 14, wherein the extension blocker is a 3′ carbon-based spacer such as C3, C6, or C12 or a 3′ quencher such as the black hole quencher.

16. The method of any one of aspects 1 to 15, wherein the PBNJ contains a locked nucleic acid (LNA) at a SNP position.

17. The method of any one of aspects 1 to 16, wherein the PBNJ has:
a length of between 10 and 50 nucleotides;
a target sequence complementarity to at least a portion of the target sequence that is between 90% and 95%; and
a reference sequence complementarity that is greater than the target sequence complementarity so that:
   the binding affinity of the PBNJ to the reference sequence is greater than a binding affinity of the PBNJ to the target sequence;
   the binding affinity of the PBNJ to the reference sequence is greater than a binding affinity of the labeled probe to the reference sequence; and/or
   the binding affinity of the PBNJ to the target sequence is less than a binding affinity of the labeled probe to the target sequence.

18. The method of any of aspects 1 to 17, comprising a plurality of PBNJ's that specifically bind to every possible SNP at a specific location in the target sequence.

19. The method of any of aspects 1 to 18, wherein the target sequence is between 10 and 50 nucleotides in length and the probe and the PBNJ are each between 10 and 50 nucleotides in length.

20. The method of any one of aspects 1 to 19, used in a biological sample to test for mutations associated with an elevated risk or presence of cancer.

21. The method of any one of aspects 1 to 19, used in a biological sample to test for a variant of a pathogen, including a pathogen that is a virus, a bacteria, or a fungus.

22. The method of any one of aspects 1 to 21, wherein the biological sample is from wastewater, environmental sample, bodily fluid, tissue, cell culture, or tumor.

23. The method of any of aspects 1 to 22, wherein the labeled-probe has a polynucleotide sequence that differs from the PBNJ sequence by one or more nucleotides.

24. The method of any of aspects 1 to 23, wherein the PBNJ is a PCR blocker during a PCR amplification cycle to provide enrichment of a target sequence that is part of a mutant sequence.

25. The method of any of claims 1 to 24, wherein a ratio of PBNJ concentration to labeled probe concentration is: equimolar or greater (e.g., 1:1 to 16:1); or less than equimolar (e.g., 0.1:1 to 0.99:1).

26: A kit for discriminating a target sequence from a reference sequence in a biological sample by polymerase chain reaction (PCR), the kit comprising:

a forward and reverse primer useful for amplifying both reference and target strands;
a labeled probe comprising a fluorophore and a quencher that specifically binds to the target sequence and may non-specifically bind to the reference sequence;
a promiscuity-blocking nucleotide juror oligonucleotide (PBNJ) that specifically binds to the reference sequence and may non-specifically bind to the target sequence;
optionally, a positive control for the reference sequence; and
optionally a positive control for the target sequence.

27: The method or kit of any of the aspects 1-26, wherein:

a forward primer is provided at a concentration of between 200 nM and 1100 nM;
a reverse primer is provided at a concentration of between 200 nM and 1100 nM;
the labeled-probe is provided at a concentration of 50-800 nM;
the PBNJ is provided at a concentration that is between 0.25× and 16× the concentration of the labeled-probe.

28. The kit of aspect 26 or 27, further comprising reagents for dPCR, qPCR, RT-dPCR or RT-qPCR.

29. The kit of any one of aspects 26 to 28, wherein assay reagents are provided for a first reaction not comprising a PBNJ and a second reaction comprising at least one PBNJ.

30. The kit of any one of aspects 26 to 29, wherein a PBNJ is provided to the wild-type reference sequence of a SARS-CoV-2 mutation selected from the group consisting of spike residues HV69-70, R408, K417, L452, T478, N501, N679, L704, Q954, and L981, and optionally also from K417, 478 and L452.

31. The method or kit of any one of aspects 1-30, wherein the reference sequence is:
a. a parental SARS-CoV-2 and the target sequence comprises a variant of SARS-CoV-2 selected from the group consisting of the Alpha variant, Beta variant, Gamma variant, Delta variant, Delta Plus variant, Mu variant, Lambda variant, Omicron variant; or
b. a proto-oncogene and the target sequence has a mutation that converts the proto-oncogene to an oncogene indicative of a higher risk of developing cancer or presence of cancer.

32. The method or kit of aspect 31, wherein the reference sequence is a proto-oncogene.

33. The method or kit of aspect 32, wherein the proto-oncogene is KRAS.

34. The method or kit of aspect 32, wherein the target is a KRAS mutation selected from G12C, G12A, G12D, G12R or G13D.

35. The method or kit of any one of aspects 1-34, wherein the PBNJ is provided at a concentration so that one or more non-specific amplification population is optically indistinguishable from a negative partition population.

36. The method or kit of any one of aspects 1-35, wherein the PBNJ is provided at a concentration so that a threshold cut-off value for detection of the target sequence is reduced relative to the threshold cut-off value for a method or kit that does not have the PBNJ.

37. The method or kit of any of aspects 1-36, wherein the PBNJ has a length of between 80% to 100% of the labeled probe.

The assays and components described herein may be used with conventional digital PCR platforms, including the QIAGEN QIAcuity®. One digital PCR system or BIO-RAD QX200 Droplet Digital PCR System and quantitative PCR systems, including BioRad CFX96 and Thermo Scientific QuantStudio5. The assays and components described herein may be used with conventional RT-PCR platforms.

Also provided herein are compositions of matter useful for carrying out any of the methods described herein, including any one or more PBNJs with or without LNA, with or without quenchers and other components, such as one or more of primers, probes and PCR solutions, singly or in combination (see, e.g., Tables 1-6 for representative sequences, with and without LNA (indicated by +), extensions (e.g., C3, etc.). quenchers and/or optically-detectable tags.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 shows reagents comprising a reference sequence (“WT template”), a target sequence (“Mutant template”), a primer pair, a dual labeled probe and a promiscuity-blocking nucleotide juror oligonucleotide (“PBNJ”). FIG. 1 further shows a PCR reaction with those same reagents and the reduced affinity of the labeled probe to the reference sequence in the presence of the PBNJ as well as the reduced affinity of the PBNJ to the target sequence in the presence of the labeled probe.

FIGS. 2A-2B show amplification results as detected by fluorescence signal. FIG. 2A shows specific amplification and non-specific amplification results, wherein the probe binds both on-target and off-target molecules in the reaction. FIG. 2B illustrates improved detection of specific amplification, wherein there is an extinction of the non-specific signal with PBNJ.

FIGS. 3A-3C show amplification results as detected by fluorescence signal, in the presence of the PBNJ, including multiplexed results. FIG. 3A shows specific amplification for specific signal A, including an absence of non-specific amplification. FIG. 3B shows specific amplification for specific signal B, including an absence of non-specific amplification. FIG. 3C shows specific amplification for specific signal A, specific amplification for specific signal B, demonstrating amplitude tuning for multiplex strategies using a single channel, including an absence of non-specific amplification.

FIGS. 4A-4C show amplification results as detected by fluorescence signal, in the presence of the PBNJ, including multiplexed results. FIG. 4A shows amplification for two probes in the same channel. FIG. 4B shows amplification for two probes in the same, alternative channel. FIG. 4C shows amplitude tuning for multiplex strategies using several channels and 2-Dimension based analysis.

FIGS. 5A-5D show that concentrations of PBNJ relative to dual-labeled probe to the 484K mutation of SARS CoV-2 reduce non-specific signal at concentration ratios of PBNJ:probe between 2:1 and 8:1. In FIG. 5A, [PBNJ]=0 and non-specific signal is indicated by arrows. In FIG. 5B, [PBNJ]=400 nM. In FIG. 5C, [PBNJ]=800 nM. In FIG. 5D, [PBNJ]=1600 nM.

FIG. 6 shows titration of a PBNJ to reduce fluorescence levels of off-target amplification using SARS-CoV-2 variant mutation assays allowing improved discrimination of the 417N mutation.

FIG. 7 shows PBNJs being used to reduce fluorescence levels of off-target amplification of oncogenic KRAS allowing improved discrimination of allele G12C.

FIG. 8 shows channel assignment for the detection of various SARS-CoV-2 variants, in either of a first reaction or a second reaction.

FIG. 9 shows an example of RT-qPCR utility for PBNJ in SARS-CoV-2 variant discrimination. As seen in the upper panel, the HEX (upper curve) traces from the left-hand amplification curves represent nonspecific 484Q probe hybridization on the on E484 template. As seen on the lower panel, addition of PBNJ completely inhibits nonspecific probe hybridization.

FIG. 10 summarizes the genes and cancer-associated mutations relevant for the PBNJ minimum requirement analysis.

FIGS. 11A-11B illustrate the impact of temperature, sequence length and LNA presence on specificity. FIG. 11A Effect of temperature (Tₘ) on target specificity in the absence of a PBNJ for KRAS-G12C (FAM, blue; top panel) and EGFR-T790M (HEX, green; bottom panel) on mixed WT and mutant synthetic DNA. Specificity is achieved only at high Tₘ but at the expense of PCR efficiency and signal to noise ratio. FIG. 11B illustrates the effects of PBNJ length, with and without an LNA at the location of the SNP, at 14× concentration and annealing/extension at 59° C. A PBNJ length of at least 80% the length of the probe improves specificity and reduces non-specific signal. In all cases, LNA presence improves specificity. 100% length with an LNA achieves complete specificity and eliminates non-specific signal for both targets. Non-specific signal is denoted by the dashed boxes.

FIGS. 12A-12B illustrate impacts of PBNJ:probe ratio, length, and presence of LNA on specificity. Effects of PBNJ ratio and the presence of an LNA on KRAS-G12C (FAM, blue; top panels) and EGFR-T790M (HEX, green; bottom panels) target specificity on mixed WT and mutant synthetic DNA at 80% PBNJ length (FIG. 12A) and 100% PBNJ length (FIG. 12B). Non-specific signal is denoted by the red hatched boxes.

FIGS. 13A-13B illustrate that PBNJs do not affect target amplification on pure KRAS-G12C (FIG. 13A) or EGFR-T790M (FIG. 13B) synthetic DNA. The concentrations of target DNA are quantified. Differences in concentrations with PBNJ compared to the absence of PBNJ are not statistically different as determined by a z-test. n.s., not significant.

FIGS. 14A-14C illustrates that PBNJ-G inhibits off-target amplification for RT-PCR amplification of SARS-CoV 2 variant templates. The plots are Relative Fluorescence Units (RFU) as a function of amplification cycle number for No PBNJ (FIG. 14A), PBNJ-G C3 (FIG. 14B) and PBNJ-G BHQ-1 (FIG. 14C) for an off-target template (top panels) and on-target template (bottom panel). Both types of PBNJ inhibit off-target amplification without adversely impacting on-target amplification.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

In general, the terms and phrases used herein have their art-recognized meaning, which can be found by reference to standard texts, journal references and contexts known to those skilled in the art. The following definitions are provided to clarify their specific use in the context of the invention.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used herein, including the appended claims, the singular forms “a,” “an,” and “the” include plural references, unless the content clearly dictates otherwise, and are used interchangeably with “at least one” and “one or more.”

As used herein, the term “about” represents an insignificant modification or variation of the numerical value such that the basic function of the item to which the numerical value relates is unchanged. In certain aspects, about indicates 90% of the stated value.

As used herein, the terms “biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), dried blood spots (e.g., obtained from infants), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. In certain aspects, the biological sample is used to test for mutations associated with an elevated risk of disease. In certain further aspects, when testing for risk of disease, the reference sequence is reflective of a low-disease condition state and the target sequence has one or more nucleotide changes in the reference sequence reflective of an elevated disease condition risk or presence of disease. In certain aspects, the biological sample is used to test for mutations associated with an elevated risk of cancer, dementia and/or cardiovascular conditions. In certain aspects, the biological sample is used to test for a variant of a pathogen, including a pathogen that is a virus. In certain further aspects, when testing for a variant of a virus, the reference sequence is from a wild-type virus or a parent virus and the target sequence comprises at least one mutation in the reference sequence. In certain aspects, the biological sample is from wastewater, environmental sample, bodily fluid, tissue, cell culture, or tumor.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

As used herein, “discriminating” refers to making a distinction or distinguishing between two or more things. In certain aspects, “discriminating” refers to distinguishing between a reference oligonucleotide sequence and a target oligonucleotide sequence.

As used herein, “dPCR” refers to partitioning of samples, with each partitioned sample analyzed for presence or absence of an amplicon, with a statistical analysis of amplicon detection across the partitions to correct for multiple targets provided to individual partitions. In this manner high sensitivity and quantification is achieved. dPCR can be applied to a sample which originally comprised DNA or a sample comprising complementary DNA obtained from reverse transcription of RNA.

As used herein, “non-specifically binds” refers to binding or hybridization of a binding agent which is not correlated with the specificity of the binding agent. In certain aspects, the binding agent is an oligonucleotide which non-specifically hybridizes to an oligonucleotide sequence which is not completely complementary to the sequence of the oligonucleotide binding agent.

As used herein, “promiscuity” refers to non-specific hybridization of nucleic acids.

As used herein, “quantitative PCR”, “qPCR” or “real-time PCR” refers to a PCR-based technique that couples amplification of a target DNA sequence with quantification of the concentration of DNA in the reaction. qPCR can be applied to a sample which originally comprised DNA or a sample comprising complementary DNA obtained from reverse transcription of RNA.

As used herein, “reference sequence” refers to an oligonucleotide sequence selected as the basis for comparison to a target sequence. In certain aspects, a target sequence is a sequence which differs from a reference sequence by one or more single nucleotide polymorphism, insertion or deletion. The term reference sequence, however, is intended to be used broadly herein. For example, although use of PBNJs has applications toward the parental or wild type sequence, PBNJs can also be used for any mutation that occurs within the probe binding region. As an illustrative example, a PBNJ designed as a G12C probe that can also include a wild type PBNJ, can also include other different reference sequences, such as a G12A, G12R, G13D, etc., PBNJ. Most broadly, a “reference sequence” simply refers to a sequence with one or more nucleotide difference(s) compared to a target sequence.

As used herein, “target” or “target sequence” are used interchangeably to refer to a nucleic acid that hybridizes to a primer and can be detected and quantified by dPCR and qPCR analysis. Target or target sequence is used broadly to refer to any oligonucleotide sequence of interest, including a sequence associated with a pathogen (e.g., virus or bacteria) and a sequence associated with a patient genome (e.g., a mammal, such as a human). A labeled probe binds specifically to the target or target sequence and, therefore, has complementarity to the target or target sequence. In certain aspects, a target or target sequence is an oligonucleotide sequence which differs from a selected reference sequence by one or more single nucleotide polymorphism, insertion or deletion. In certain aspects, the target or target sequence is between 10 and 50 nucleotides in length. In certain further aspects, the target or target sequence is a region of 10 and 50 nucleotides, within a polynucleotide of substantially longer length.

A target or target sequence in the presence of corresponding primers and probes, polymerase, optionally reverse transcriptase, nucleotides, and at a suitable pH, temperature, metal and ion concentration, will specifically hybridize to a single-stranded target sequence and initiate synthesis of a second strand complementary to the target. This amplification may be repeated by cycling of temperature for repeated hybridizing and separation, thereby amplifying any target sequences.

A primer does not need to reflect the exact sequence of the template but must be sufficiently complementary to hybridize with a template. A primer may further comprise a “tail” comprising additional nucleotides at the 5′ end of the primer that are non-complementary to the template. Typically, the lengths of primers range between 7-100 nucleotides in length, such as 10-30, 15-60, 20-40, and so on, more typically in the range of between 15-35 nucleotides in length, and any sub-ranges thereof. Shorter primer molecules generally require cooler temperatures to form sufficiently stable hybrid complexes with the template. The term “primer site” or “primer binding site” refers to the segment of the target DNA to which a primer hybridizes. Typically, a set of primers is used for amplification of a target polynucleotide, including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. Useful primers may be designed in accordance with any of the teachings provided in U.S. Pat. No. 10,465,238, which is specifically incorporated by reference herein, including for different 5′ tail lengths to facilitate amplicon differentiation from a first target and a different target based on amplicon length.

As used herein, “sequence complementarity” refers to the standard arrangement of bases in nucleotides in relation to their opposite pairing, such as thymine being paired with adenine and cytosine being paired with guanine. In certain aspects, sequence complementarity is complete or exact complementarity at all base positions within an oligonucleotide. RNA has uracil instead of thymine.

As used herein, “specifically binds” refers to hybridization between complementary oligonucleotides or sequences of nucleotides. In certain aspects, a probe is designed to be specific to a target region wherein the probe has a sequence which is complementary to the nucleotide sequence of the target region. In an aspect, the probe may be completely (e.g., 100%) complementarity to the target.

As used herein, “suppresses labeled probe” refers to competition by a competitor (e.g., PBNJ) with probe binding at a hybridization region which is not completely complementary to the probe, thereby reducing such non-specific probe binding and detection of a signal from such non-specific probe binding.

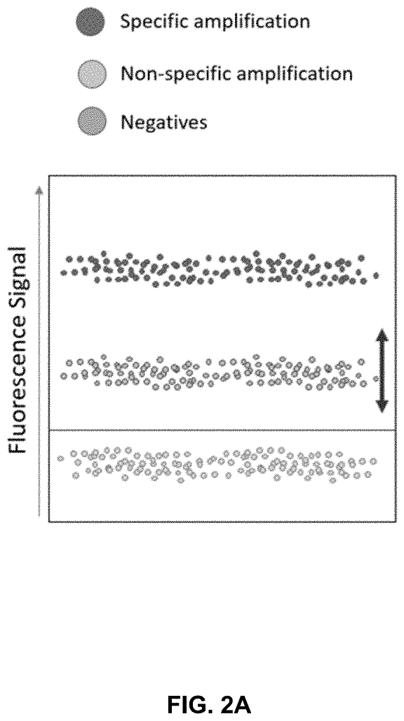

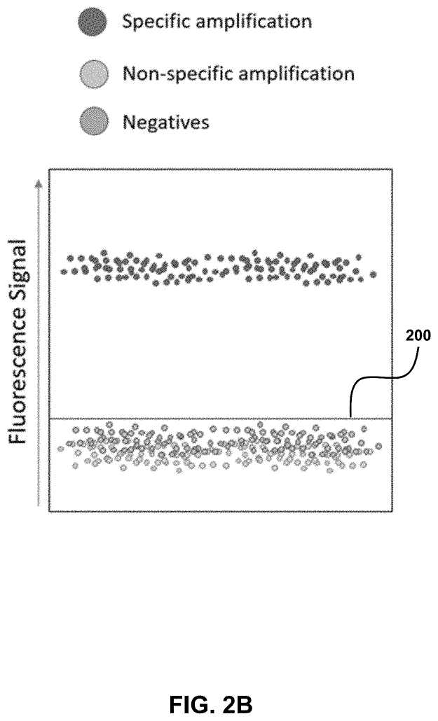

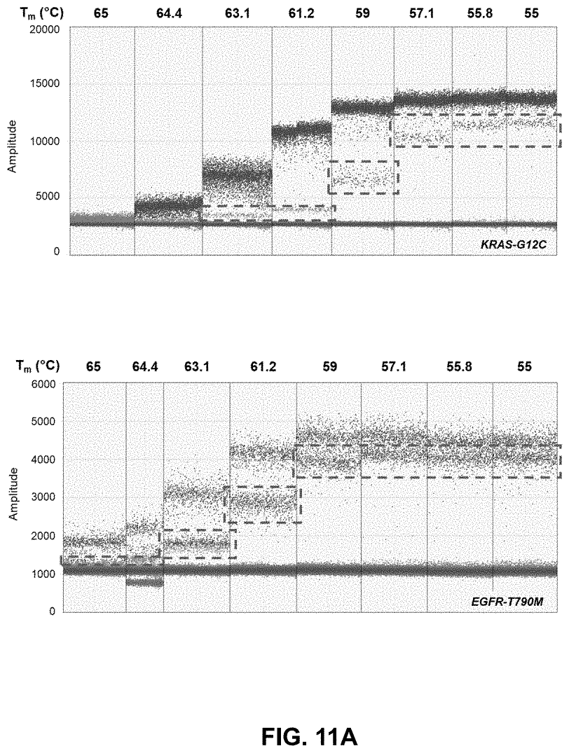

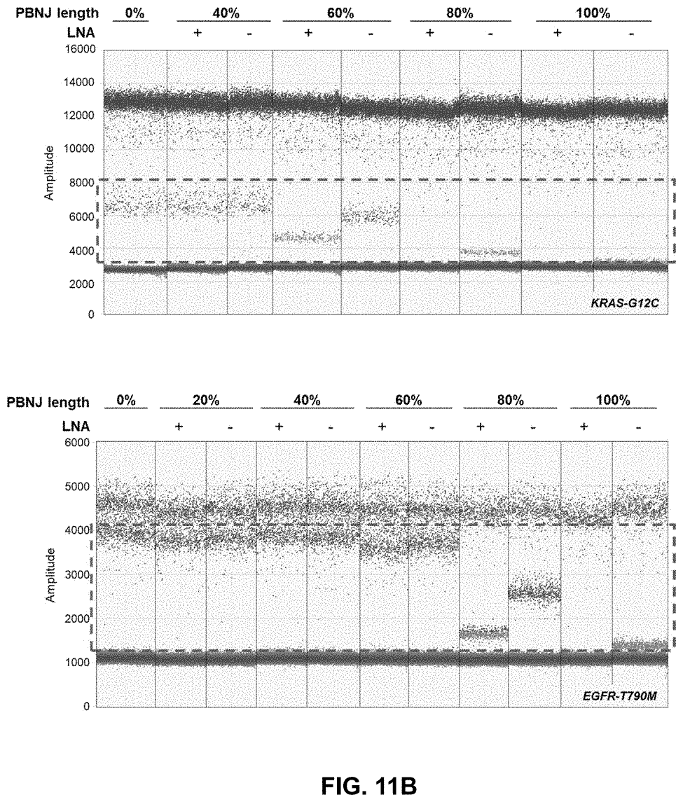

As used herein, “substantially eliminates” refers to a reduction of at least 90%, at least 95%, or at least 99%. In the context of using thresholds, wherein different populations are defined by the setting of thresholds (see, e.g., the dashed boxes in FIGS. 11-13, and line 200 in FIG. 2B and similar other lines in the Figures delineating populations), even a PBNJ knock-down of signal by 20% can be sufficient to reliably distinguish target from off-target.

The present disclosure provides methods for discriminating a target sequence from a reference sequence in a biological sample by PCR. In certain aspects, the PCR is dPCR. In certain further aspects, the discrimination between the target and the reference sequence is achieved by introduction to a PCR reaction of a promiscuity-blocking nucleotide juror oligonucleotide (PBNJ) that specifically binds to the reference sequence and may non-specifically bind to the target sequence, wherein the PBNJ comprises a reference binding region and an extension blocker that prevents elongation by a polymerase.

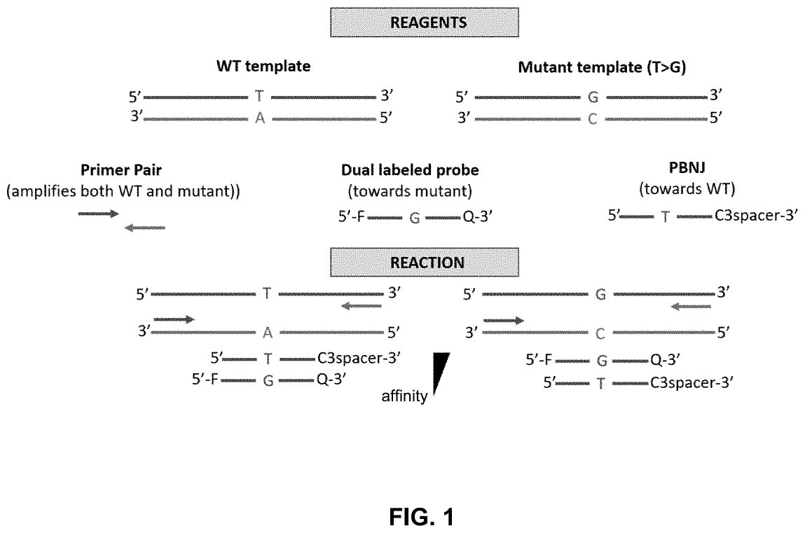

As shown in FIG. 1, the PBNJ can compete for binding to the reference sequence with a labeled probe which is specific to a target sequence but able to non-specifically bind to a reference sequence. The reduced affinity of the labeled probe to the reference sequence, provided by PBNJ competition, reduces error signal produced by non-specific binding of the probe to the reference sequence.

In certain aspects, the PBNJ sequence is similar to the sequence of a probe oligonucleotide labeled with both a fluorophore and a quencher, i.e. a dual labeled probe. As shown in FIG. 1, in certain aspects only the single nucleotide polymorphism (SNP) base is changed (SNP can be located either internally or at the 3′-end). Thus, the target and reference sequence can have a single nucleotide mismatch that is a single nucleotide polymorphism (SNP) or is part of a short nucleotide polymorphism, such as a deletion or insertion.

In certain aspects, the probe labeled with both a fluorophore and a quencher is a conventional probe used in qPCR, containing a fluorophore (F) at the 5′-end and usually a quencher (Q) at the 3′ end, see FIG. 1. In certain aspects, the probe is 10 to 50 nucleotides in length.

In certain aspects, the PBNJ has a C3-spacer modification at the 3′-end to prevent 5′ to 3′ elongation by a DNA polymerase. In certain further aspects, the PBNJ and the probe compete during hybridization, increasing target specificity of the probe due to change in affinity for respective targets as shown in FIG. 1, particularly for PBNJ in excess. In certain aspects, the modification at the 3′-end of the PBNJ is a C3-spacer or a 3′ quencher such as a black hole quencher. The PBNJ can also be modified with extended carbon-spacers (C6, C9, C12, et). The PBNJ can have any length useful in the present methods, including but not limited to a length of 10 to 50 nucleotides. In certain aspects, the PBNJ has a target sequence complementarity to at least a portion of the target sequence that is less than or equal to a range that is between 9/10 nucleotides and 49/50 nucleotides (e.g., one or greater mismatch over the length of the PBNJ). In certain aspects, a PBNJ has a reference sequence complementarity that is greater than the target sequence complementarity such that: a binding affinity of the PBNJ to the reference sequence is greater than a binding affinity of the PBNJ to the target sequence; and the binding affinity of the PBNJ to the reference sequence is greater than a binding affinity of the labeled probe to the reference sequence; and/or the binding affinity of the PBNJ to the target sequence is less than a binding affinity of the labeled probe to the target sequence. In certain aspects, the PBNJ is a PCR blocker during a PCR amplification cycle to provide enrichment of a target sequence that is part of a mutant allele. In certain aspects, a PBNJ may have modifications at the 5′-end to increase its stability by preventing digestion by a DNA polymerase.

In certain aspects, the ratio of probe to PBNJ is such that the concentration of PBNJ is in excess of the concentration of the probe. In certain aspects, a ratio of PBNJ concentration to dual-labeled probe concentration is at least 1.5:1. In certain further aspects, the PBNJ acts as a PCR blocker during amplification cycles, leading to mutant allele enrichment. In certain aspects, reactions can be multiplexed to utilize two or more PBNJs.

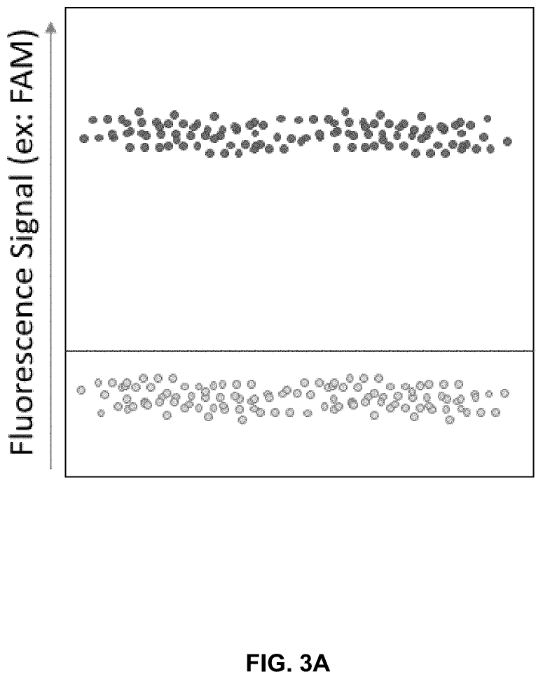

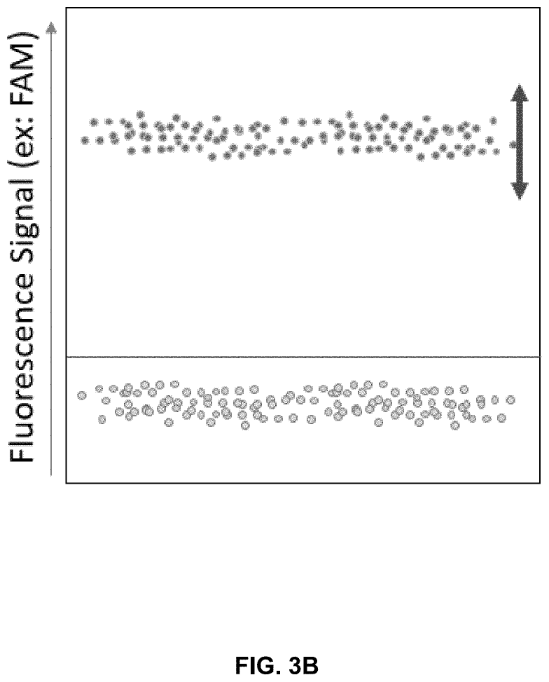

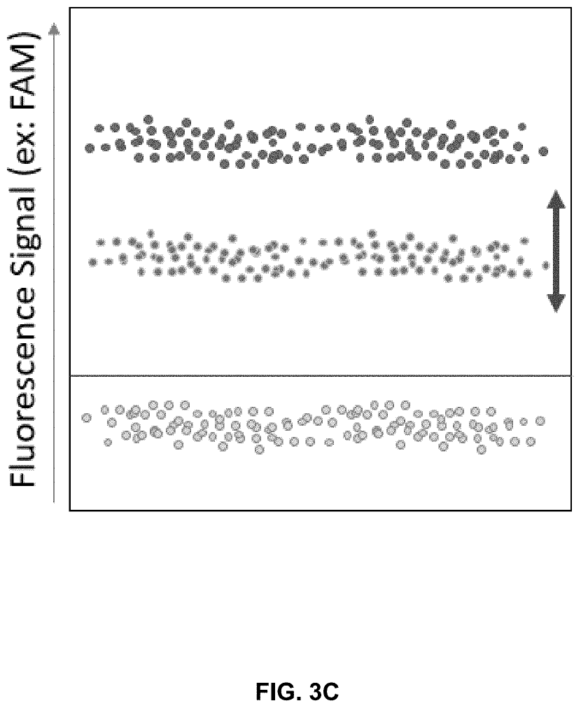

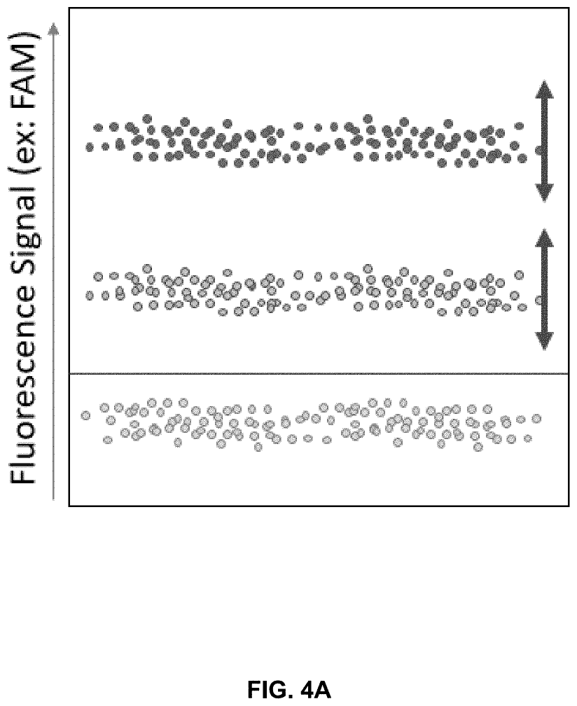

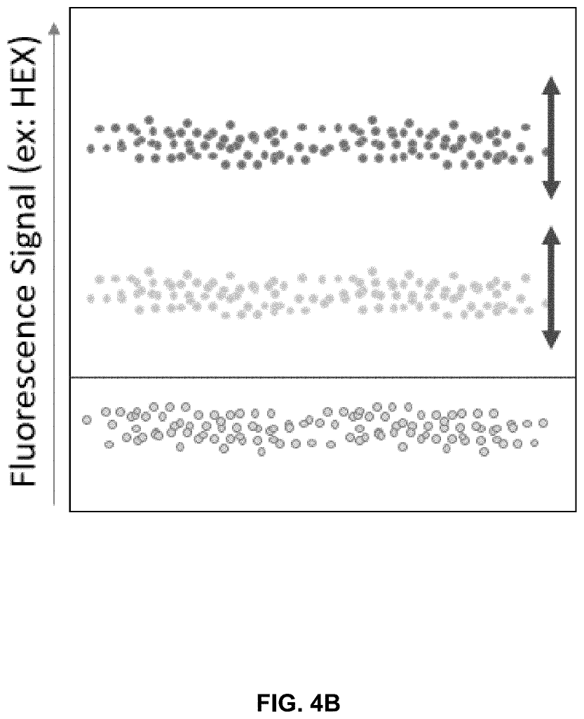

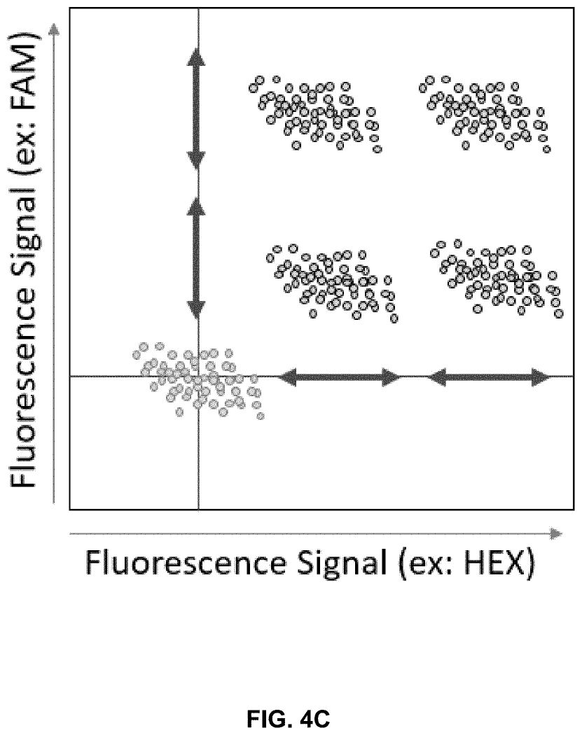

One practical impact of the methods, kits, and compositions provided herein is illustrated in FIGS. 2A-4C. In particular, use of PBNJ effectively reduces detection of undesired amplicons show amplification results as detected by fluorescence signal (compare FIG. 2A without PBNJ and FIG. 2B with PBNJ). Possibly interfering signal associated with non-specific amplification is reduced by use of the PBNJ provided therein. This improves the ability to properly set a threshold 200. FIG. 2A shows specific amplification and non-specific amplification results, wherein the probe binds both on-target and off-target molecules in the reaction. FIG. 2B illustrates improved detection of specific amplification, wherein there is an extinction of the non-specific signal with PBNJ.

In certain aspects, a reference sequence and a target sequence differ by a single nucleotide; a nucleotide insertion of one or more nucleotides; and/or a nucleotide deletion of one or more nucleotides.

The methods and kits disclosed herein can be utilized with any oligonucleotide, including but not limited to DNA and/or RNA. In certain aspects, the methods and kits disclosed herein are applied for dPCR, qPCR, RT-dPCR and RT-qPCR.

In certain aspects the labeled probe is a single-nucleotide variant (SNV)-specific TaqMan® probe (a fluorophore covalently attached to a 5′ end of the probe and a quencher at a 3′ end of the probe).

In certain aspects, the dPCR comprises partition and droplet-based PCR and the PBNJ eliminates signal associated with a lower efficiency, non-specific off-target amplification, thereby increasing a signal to noise ratio for specific amplification of the target sequence.

In certain aspects, the probe output amplitude is tuned by providing the PBNJ at a lower concentration. In certain further aspects, a plurality of probe output amplitudes is tuned for multiplex detection of a plurality of target sequences in a single or a multichannel fluorescence detector.

In certain aspects, the method or kit disclosed herein is designed for a reference sequence which is a parental SARS-CoV-2 and a target sequence comprises a variant of SARS-CoV-2, such as Alpha, Beta, Gamma, Delta, and Omicron. In certain aspects, the method or kit disclosed herein is designed for a reference sequence which is a proto-oncogene and a target sequence which has a mutation that converts the proto-oncogene to an oncogene indicative of a higher risk of developing cancer or presence of cancer.

In certain aspects, the method or kit disclosed herein provides a PBNJ at a concentration so that one or more non-specific amplification population is optically indistinguishable from a negative partition population.

Kits are disclosed for practicing the methods disclosed herein. Kits provide for discriminating a target sequence from a reference sequence in a biological sample by dPCR or RT-PCR. In certain aspects, kits comprise buffers, primers, polymerase, one or more labeled probes and one or more PBNJ's. In certain aspects, a forward primer is provided at a concentration of between 50 nM and 1100 nM. In certain aspects, a reverse primer is provided at a concentration of between 50 nM and 1100 nM. In certain aspects, labeled-probe is provided at a concentration of 20-800 nM. In certain aspects, one or more PBNJ is provided at a concentration that is between 0.25× and 16× the concentration of the labeled-probe.

In one aspect, a kit is provided comprising 200× 26 k reactions (400× 8.5 k partition reactions) per Assay solution. In certain further aspects, the kit contains all primers, probes, and controls for detection and discrimination of Alpha, Beta, Gamma, Delta, Delta Plus, Mu and Lambda variants of SARS-CoV-2 in two multiplexed dPCR wells.

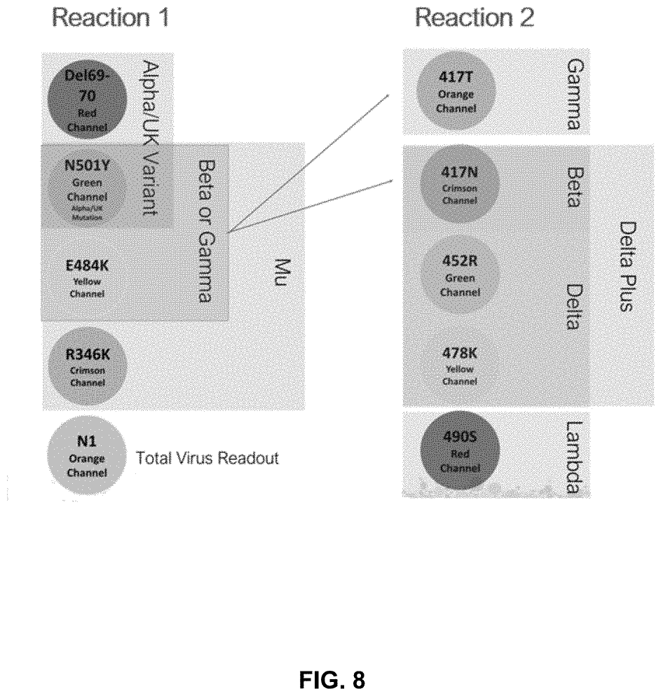

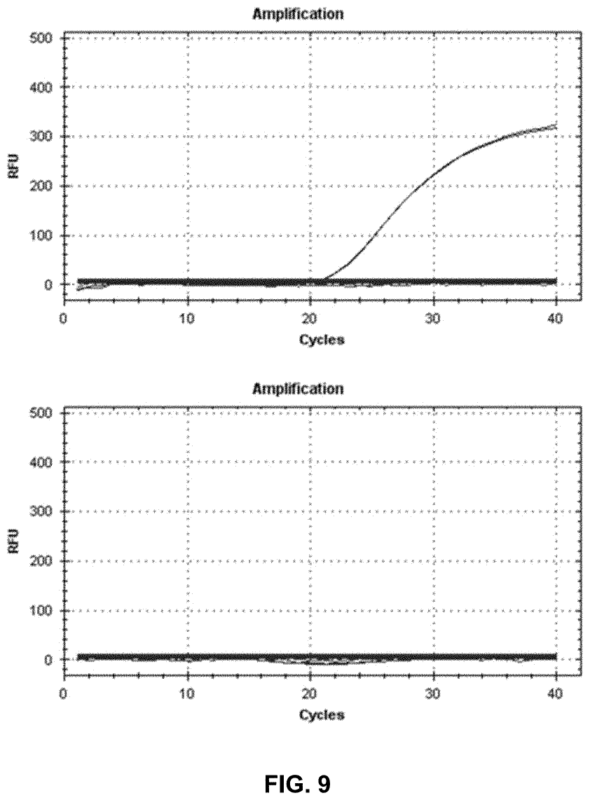

In certain further aspects, a kit contains reagents for reactions as depicted in FIG. 8, with oligonucleotides comprising sequences as specified in Table 1. In certain further aspects, the kit comprises Reaction 1 Assay Solution—20× (400 uL); Reaction 2 Assay solution—20× (400 uL); Parental (non-variant/Wuhan) positive control—enough for at least 20 tests and Mixed Variant Positive Control containing all 7 variant templates—enough for at least 20 tests.

In certain aspects, as shown in FIG. 8, channel assignments can be made as in a first reaction (Reaction 1) and/or a second reaction (Reaction 2). Table 1 provides sequences useful, according to the methods disclosed herein, for Reaction 1 or Reaction 2, as in FIG. 8. Controls for reaction 1 can include the parental SARS-CoV-2 positive control. Controls for reaction 2 can include an Alpha variant positive control, a Delta variant positive control, a Delta Plus variant positive control, a Mu variant positive control, a Lambda variant positive control, a Gamma variant positive control and a Beta variant positive control. Unless noted otherwise, the symbol + refers to the position of the LNA (with +A referring to the LNA on the adenine).

TABLE 1
SARS-COV-2 Variant Assay Panel
Oligonucleotide Sequences

| SEQ ID | | | |
|---|---|---|---|
| NO: | Oligo ID | Sequence | Rxn |
| 1 | 452_fwd1 | TGCGTTATAGCTTGGAATTCT | 2 |
| | | AACAATC | |
| 2 | 484-501_rev2 | TTGCTGGTGCATGTAGAAG | 2 |
| 3 | 417 fwd2 | TGTAATTAGAGGTGATGAAGT | 2 |
| | | CAGAC | |
| 4 | 417 rev | CAAGCTATAACGCAGCCTG | 2 |
| 5 | 69-70 fwd1 | CGTGGTGTTTATTACCCTGAC | 1 |
| 6 | 69-70 rev3 | CACCATCATTAAATGGTAGGA | 1 |
| | | CAG | |
| 7 | 417T_RT1_TAM | CAAACTGGAACGATTGCTGAT | 2 |
| 8 | 417N_RT1_Cy5 | CCAGGGCAAACTGGAAATATT | 2 |
| | | G | |
| 9 | 501Y_5-FAM | TTTCCAACCCACTTATGGTGT | 1 |
| | | TG | |
| 10 | del69-70_3_TX | TGGTTCCATGCTATCTCTGG | 1 |
| | Red | | |
| 11 | 484K_RT1-HEX | CACACCTTGTAATGGTGTTAA | 1 |
| | | AGG | |
| 12 | N1-fwd | GAC CCC AAA ATC AGC | 1 |
| | | GAA AT | |
| 13 | N1-rev | TCT GGT TAC TGC CAG | 1 |
| | | TTG AAT CTG | |
| 14 | N1-TAMRA | ACC CCG CAT TAC GTT | 1 |
| | | TGG TGG ACC | |
| 15 | 346 fwd | ACT TGT GCC CTT TTG | 2 |
| | | GTG AAG | |
| 16 | 346 rev | TAG GAC AGA ATA ATC | 2 |
| | | AGC AAC ACA GT | |
| 17 | 346K-RT3-Cy5 | ACG CCA CCA +AAT TTG | 2 |
| | | C | |
| 18 | 490S_RT2-TX | TGT TGA AGG TTT TAA | 2 |
| | Red | TTG TTA CTC TCC | |
| 19 | 346_S-PBNJ | ACG CCA CCA GAT TTG | 1 |
| | | C-CH2-CH2-CH3 | |
| 20 | 417_RT-PBNJ | CCAGGGCAAACTGGAAAGATT | 2 |
| | | G-CH2-CH2-CH3 | |
| 21 | 478_RT-PBNJ | CAA CAC CAT TAC AAG | 2 |
| | | GTG TGC TAC-CH2-CH2- | |
| | | CH3 | |
| 22 | 478K_RT2-HEX | CAACACCATTACAAGGTTTGC | 2 |
| | | TAC | |
| 23 | 452R_RT2-FAM | AATTACCGGTATAGATTGTTT | 2 |
| | | AGGAAGTC | |
| 24 | 478K-RT4- | ACA CCA TTA CAA GGT | 2 |
| | HEX | TTG CTA C | |

### EXAMPLES

The following examples are provided for illustrative purposes only and are not intended to limit the scope of the application as defined by the appended claims. All examples described herein were carried out using standard techniques, which are well known and routine to those of skill in the art. Routine molecular biology techniques described in the following examples can be carried out as described in standard laboratory manuals, such as Sambrook et al., Molecular Cloning: A Laboratory Manual, 3rd. ed., Cold Spring Harbor Laboratory Press, Cold Spring Harbor, N.Y., (2001).

### Example 1: Reduction of Off-Target E484 Signal with PBNJ

This example describes the use of PBNJs to reduce off target amplification using E484K probe. Dual-labeled probe was designed towards the mutant 484K sequence. The concentrations of reagents utilized were as follows: common forward primer—500 nM; common reverse primer—500 nM; dual-labeled probe to 484K—200 nM; and PBNJ concentration was variable. Assays were conducted on the QIAGEN—QIAcuity® instrument.

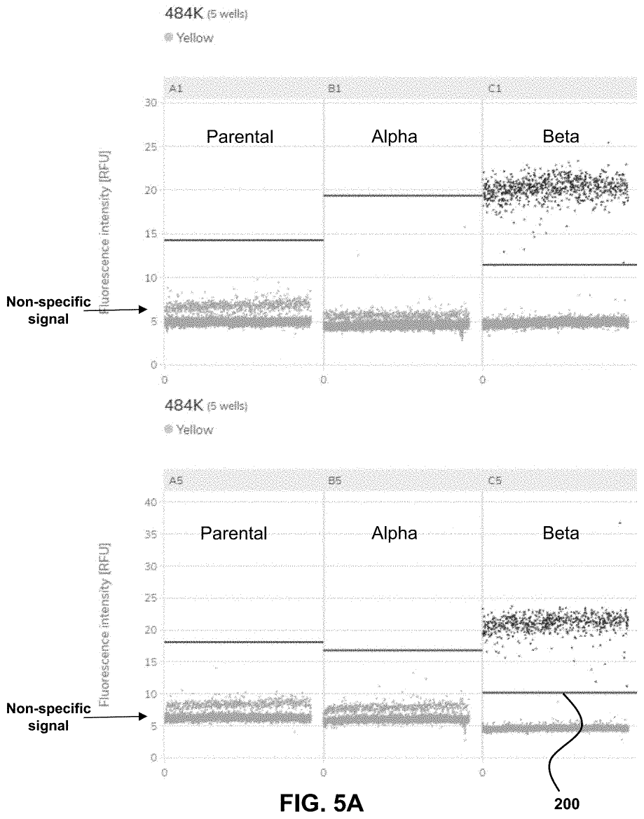

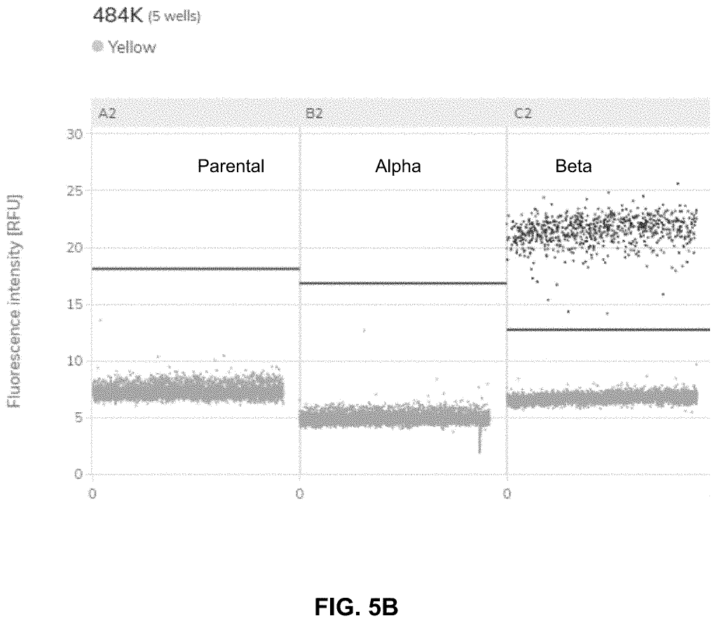

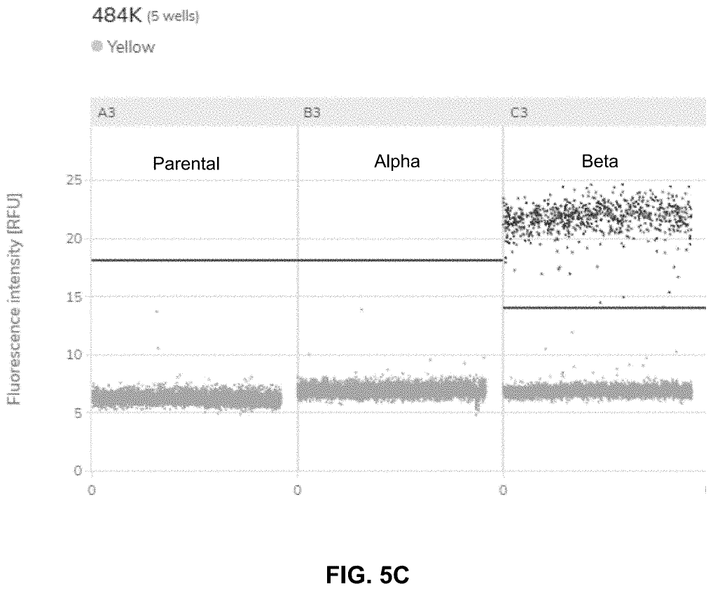

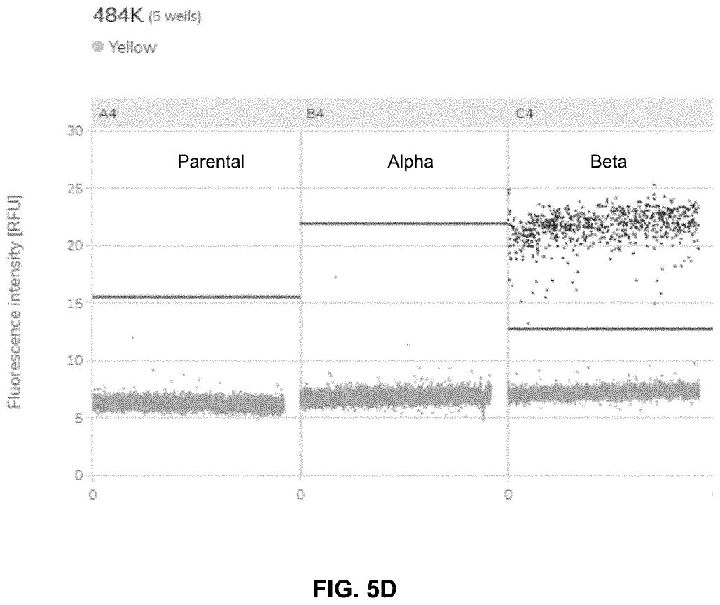

FIG. 5A shows that the probe detects 484K in the “Beta” SARS-CoV-2 variant but results in off-target amplification, in the absence of PBNJ (i.e. [PBNJ]=0). The arrow in FIG. 5A indicates the secondary low efficiency, lower amplitude population in “Parental/wildtype” and “Alpha” controls which are wild-type at the 484 locus.

FIG. 5B-5D show results for increasing concentrations of PBNJ. FIG. 5B shows that off-target E484 signal is extinct when PBNJ is added at a competitive concentration of 400 nM, which corresponds to a ratio of [2:1] (PBNJ:Probe). FIG. 5C shows similar results at [PBNJ]=800 nM. FIG. 5D shows similar results at [PBNJ]=1600 nM. Thus, excess PBNJ (up to ratio 8:1) does not affect on-target detection, while extinguishing non-specific signal.

### Example 2: Reduction of Off-Target 417N Signal with PBNJ

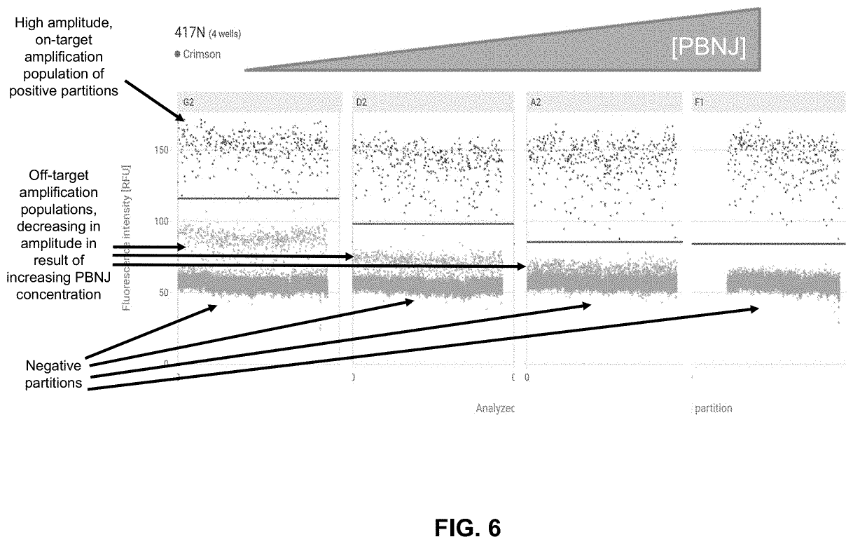

This example describes the use of PBNJs to reduce off target amplification using 417N probe. Dual-labeled probe was designed towards the mutant 417N sequence. The dual labeled probe was used on a mixture of template consisting of Parental, Beta, Gamma, and Delta variants of SARS-CoV-2. In well G02, the probe produced a high amplitude, population of droplets resulting from on-target amplification on template from the beta variant of SARS-CoV-2 which contains the 417N mutation (highlighted by arrow in FIG. 6) and off-target, lower amplitude populations of partitions due to off-target, less efficient amplification. Assays were conducted on the QIAGEN—QIAcuity® instrument.

Upon titration of a PBNJ designed to contain sequence complimentary to the wild-type K417 allele, the amplitude of the off-target amplification populations (highlighted by arrows in FIG. 6) decreased until the non-specific amplification populations were non-distinguishable from the negative droplets (also highlighted by arrow in FIG. 6).

### Example 3: Reduction of Off-Target Human KRAS G12C Signal with PBNJ

This example describes the use of PBNJs to reduce off target amplification using KRAS G12C probe. Dual-labeled probe was designed towards the mutant KRAS G12C sequence. The concentrations of reagents utilized were as follows: common forward primer—900 nM; common reverse primer—900 nM; dual-labeled probe to G12C—250 nM; and PBNJ—500 nM. Assays were conducted on the BIORAD—DROPLET DIGITAL QX200 instrument. See Tables 2-5 for sequence information and concentrations tested.

TABLE 2
KRAS Human Gene Sequence and Variants

| | Partial Human KRAS |
|---|---|
| | Gene Sequence 5′→3′ |
| “Wild-type” | ... A TAT AAA CTT GTG GTA GTT |
| gene sequence | GGA GCT GGT GGC GTA GGC AAG |
| | AGT GCC TTG ACG ATA CAG CTA |
| | ATT CAG AAT C ... (SEQ ID |
| | NO: 25) |
| G12C variant | ... A TAT AAA CTT GTG GTA GTT |
| | GGA GCT TGT GGC GTA GGC AAG |
| | AGT GCC TTG ACG ATA CAG CTA |
| | ATT CAG AAT C ... (SEQ ID |
| | NO: 26) |
| G12R variant | ... A TAT AAA CTT GTG GTA GTT |
| | GGA GCT CGT GGC GTA GGC AAG |
| | AGT GCC TTG ACG ATA CAG CTA |
| | ATT CAG AAT C ... (SEQ ID |
| | NO: 27) |

TABLE 3
Probes, PBNJ and Primers-KRAS G12C Variant,
KRAS G12R Variant, KRAS wild-type

| | Probe | Sequence | 5′- | internal | 3′- | |
|---|---|---|---|---|---|---|
| Oligo Type | Name | 5′→3′ | mod′n | mod′n | mod′n | Orient. |
| TaqMan probe | Probe | CC TAC GCC | FAM | ZEN | 3IABkFQ | − |
| (SEQ ID NO: | G12C | ACA AGC TCC | | | | |
| 28) | rev | AAC TA | | | | |
| Competitor | Kras | CC TAC GCC | — | — | C3-spacer | − |
| (SEQ ID NO: | PBNJ | ACG AGC TCC | | | | |
| 29) | G12R | AAC TA | | | | |
| Forward | KRAS | TAAGGCCTGCT | — | — | — | + |
| Primer | G12C | GAAAATGACTG | | | | |
| (SEQ ID NO: | Fwd | | | | | |
| 30) | | | | | | |
| Reverse | KRAS | GGTCCTGCAC | — | — | — | − |
| Primer | G12C | CAGTAATATGC | | | | |
| (SEQ ID NO: | Rev | | | | | |
| 31) | | | | | | |

TABLE 4
Useful Concentrations of Probes, PBNJ and Primers

| | | Final | Ratio |
|---|---|---|---|
| Oligonucleotide | Probe | Concentration | Competitor: |
| Type | Name | (nM) | Probe |
| TaqMan probe | Probe G12C rev | 250 | — |
| Competitor | Kras PBNJ G12R | 500 | 2:1 |
| Forward Primer | KRAS G12C Fwd | 900 | — |
| Reverse Primer | KRAS G12C Rev | 900 | — |

TABLE 5
Useful Concentrations of Template

| DNA templates | Template description | Amount |
|---|---|---|
| G12C template | Synthetic G12C DNA gBlock | 500 copies |
| WT template | Genomic Wild-Type DNA | 1000 copies |
| G12R/WT template | genomic DNA (mixture | 1000 copies |
| | 50% WT and 50% G12R) | |

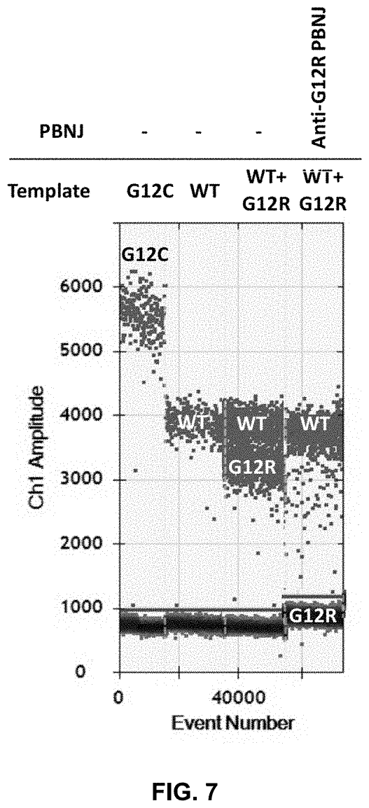

The probe produced a high amplitude population of droplets on G12C mutation containing template. This probe also produced lower amplitude off-target droplet populations on WT template and G12R template as demonstrated by the presence of lower amplitude, off target populations in wild-type template (B01, E01) and in alternative G12R mutation containing template (E01), as shown in FIG. 7.

Using a PBNJ designed to contain sequence complimentary to the G12R template, the G12R off target, lower amplitude population of droplets is abolished (D01). A secondary PBNJ complimentary to the wild-type sequence at this locus could be used to abolish that off-target lower amplitude population of droplets as well. This use shows that PBNJs can be designed to selectively remove off-target populations of the user's preference.

### Example 4: Characterization of PCR and PBNJ Parameters on Target Specificity (Cancer-Related Mutation)

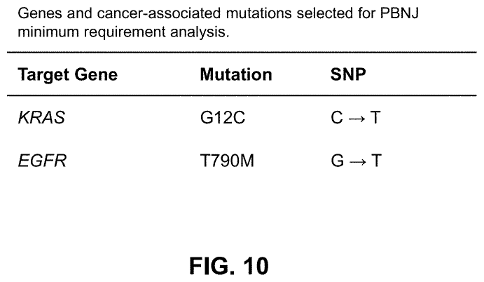

FIG. 10 summarizes the target gene and mutations thereof relevant for cancer (exemplified herein for KRAS and EGFR) with corresponding mutation and SNP relevant for FIGS. 11A-13B. Exemplary sequences used in the methods described herein are provided in Table 6 (EGFR) and Table 7 (KRAS).

TABLE 6
Exemplary primers, probes and PBNJ
for improved detection of
mutations of EGFR and KRAS by PCR.

| | | | | |
|---|---|---|---|---|
| Oligo type | Name | Sequence (5′→3′) | Orient. | Rxn |

Gene Target: EGFR

| | | | | |
|---|---|---|---|---|
| Forward Primer | T790-Fwd | GCCTGCTGGGCATCTG | + | 1 |
| (SEQ ID NO: 32) | | | | |
| Reverse Primer | T790-Rev | TCTTTGTGTTCCCGGAC | − | 1 |
| (SEQ ID NO: 33) | | ATAGTC | | |
| TaqMan Probe | T790M-P- | /5HEX/CAGCTCATCA+T | + | 1 |
| (SEQ ID NO: 34) | LNA1 | GCAGCTCATG/3IABkFQ/ | | |
| Competitor | T790-LNA- | TGCAGCTCATCA+CGCA | + | na |
| (PBNJ) | PBNJ120 | GCTCATGCC/3SpC3/ | | |
| (SEQ ID NO: 35) | | | | |
| Competitor | T790-LNA- | CAGCTCATCA+CGCAGC | + | 1 |
| (PBNJ) | PBNJ100 | TCATG/3SpC3/ | | |
| (SEQ ID NO: 36) | | | | |
| Competitor | T790-LNA- | AGCTCATCA+CGCAGCT | + | na |
| (PBNJ) | PBNJ80 | C/3SpC3/ | | |
| (SEQ ID NO: 37) | | | | |
| Competitor | T790-LNA- | TCATCA+CGCAGCT/ | + | na |
| (PBNJ) | PBNJ60 | 3SpC3/ | | |
| (SEQ ID NO: 38) | | | | |
| Competitor | T790-LNA- | ATCA+CGCA/3SpC3/ | + | na |
| (PBNJ) | PBNJ40 | | | |
| Competitor | T790-LNA- | TCA+CG/3SpC3/ | + | na |
| (PBNJ) | PBNJ20 | | | |
| Competitor | T790- | TGCAGCTCATCACGCAG | + | na |
| (PBNJ) | PBNJ120 | CTCATGCC/3SpC3/ | | |
| (SEQ ID NO: 39) | | | | |
| Competitor | T790- | CAGCTCATCACGCAGCT | + | na |
| (PBNJ) | PBNJ100 | CATG/3SpC3/ | | |
| (SEQ ID NO: 40) | | | | |
| Competitor | T790- | AGCTCATCACGCAGCTC/ | + | na |
| (PBNJ) | PBNJ80 | 3SpC3/ | | |
| (SEQ ID NO: 41) | | | | |
| Competitor | T790- | TCATCACGCAGCT/3SpC3/ | + | na |
| (PBNJ) | PBNJ60 | | | |
| (SEQ ID NO: 42) | | | | |
| Competitor | T790- | ATCACGCA/3SpC3/ | + | na |
| (PBNJ) | PBNJ40 | | | |
| Competitor | T790- | TCACG/3SpC3/ | + | na |
| (PBNJ) | PBNJ20 | | | |
| Competitor | C797- | CTTCGGC+T+GCCTCCT/ | + | 1 |
| (PBNJ) | LNA2- | 3SpC3/ | | |
| (SEQ ID NO: 43) | PBNJ1 | | | |
| TaqMan Probe | C797SC-P- | /56FAM/CTTCGGCT+CCC | + | 1 |
| (SEQ ID NO: 44) | LNA1 | TCCTG/3IABkFQ/ | | |
| TaqMan Probe | C797SA-P- | /56FAM/CTTCGGC+AGC | + | 1 |
| (SEQ ID NO: 45) | LNA1 | CTCCTG/3IABkFQ/ | | |
| Forward Primer | S768-Fwd | CTGACGTGCCTCTCCCT | + | na |
| (SEQ ID NO: 46) | | C | | |
| Reverse Primer | S768-Rev | GTGAGGCAGATGCCCA | − | 2 |
| (SEQ ID NO: 47) | | G | | |
| Competitor | S768-LNA- | TGTCCACG+CTGGCCAT | − | 2 |
| (PBNJ) | PBNJ2 | CA/3SpC3/ | | |
| (SEQ ID NO: 48) | | | | |
| TaqMan Probe | S768I-P- | /5HEX/TGTCCACG+ATG | − | 2 |
| (SEQ ID NO: 49) | LNA1 | GCCATCA/3IABkFQ/ | | |
| Forward Primer | G719-Fwd | TGGAGAAGCTCCCAAC | + | 2 |
| (SEQ ID NO: 50) | | CAA | | |
| Reverse Primer | G719-Rev | CTTATACACCGTGCCGA | − | 2 |
| (SEQ ID NO: 51) | | ACG | | |
| Competitor | G719-LNA- | CGGAG+CCCAGCACTT/ | − | 2 |
| (PBNJ) | PBNJ1 | 3SpC3/ | | |
| (SEQ ID NO: 52) | | | | |
| Competitor | G719-LNA- | CGGAGC+CCAGCACTT | − | 2 |
| (PBNJ) | PBNJ2 | /3SpC3/ | | |
| (SEQ ID NO: 53) | | | | |
| TaqMan Probe | G719S-P- | /56FAM/TCCGGAGC+TC | − | 2 |
| (SEQ ID NO: 54) | LNA1 | AGCACTT/3IABkFQ/ | | |
| TaqMan Probe | G719C-P- | /56FAM/TCCGGAGC+AC | − | 2 |
| (SEQ ID NO: 55) | LNA1 | AGCACTT/3IABkFQ/ | | |
| TaqMan Probe | G719A-P- | /56FAM/CGGAG+GCCAG | − | 2 |
| (SEQ ID NO: 56) | LNA1 | CACTT/3IABkFQ/ | | |
| Forward Primer | L858-Fwd | GCAGCATGTCAAGATC | + | 3 |
| (SEQ ID NO: 57) | | ACAGATT | | |
| Reverse Primer | L858-Rev | CCTCCTTCTGCATGGTA | − | 3 |
| (SEQ ID NO: 58) | | TTCTTTCT | | |
| Competitor | L858-LNA- | CAGTTTGGCC+AGCCCA | − | 3 |
| (PBNJ) | PBNJ1 | A/3SpC3/ | | |
| (SEQ ID NO: 59) | | | | |
| TaqMan Probe | L858R-P- | /56FAM/TTTGGCC+CGCC | − | 3 |
| (SEQ ID NO: 60) | LNA1 | CAAA/3IABkFQ/ | | |
| Competitor | L861-LNA- | ACCCAGC+AGTTTGGCC/ | − | 3 |
| (PBNJ) | PBNJ1 | 3SpC3/ | | |
| (SEQ ID NO: 61) | | | | |
| TaqMan Probe | L861Q-P- | /5HEX/ACCCAGC+TGTTT | − | 3 |
| (SEQ ID NO: 62) | LNA1 | GGCC/3IABkFQ/ | | |

Gene Target: KRAS

| | | | | |
|---|---|---|---|---|
| Forward Primer | G12-Fwd | AGGCCTGCTGAAAATG | + | 1,2,3 |
| (SEQ ID NO: 63) | | ACTG | | |
| Reverse Primer | G12-Rev | GCTGTATCGTCAAGGCA | − | 1,2,3 |
| (SEQ ID NO: 64) | | CTC | | |
| TaqMan Probe | G12C- | /56FAM/CTACGCCAC+A | − | 1 |
| (SEQ ID NO: 65) | LNA1-FAM | AGCTCCAACTA/3IABkFQ/ | | |
| Competitor | G12-LNA- | TGCCTACGCCAC+CAGC | − | na |
| (PBNJ) | PBNJ120 | TCCAACTACC/3SpC3/ | | |
| (SEQ ID NO: 66) | | | | |
| Competitor | G12-LNA- | CTACGCCAC+CAGCTCC | − | 1 |
| (PBNJ) | PBNJ100 | AACTA/3SpC3/ | | |
| (SEQ ID NO: 67) | | | | |
| Competitor | G12-LNA- | ACGCCAC+CAGCTCCAAC/ | − | na |
| (PBNJ) | PBNJ80 | 3SpC3/ | | |
| (SEQ ID NO: 68) | | | | |
| Competitor | G12-LNA- | CGCCAC+CAGCTCC/ | − | na |
| (PBNJ) | PBNJ60 | 3SpC3/ | | |
| (SEQ ID NO: 69) | | | | |
| Competitor | G12-LNA- | CCAC+CAGC/3SpC3/ | − | na |
| (PBNJ) | PBNJ40 | | | |
| Competitor | G12-LNA- | AC+CAG/3SpC3/ | − | na |
| (PBNJ) | PBNJ20 | | | |
| Competitor | G12- | TGCCTACGCCACCAGCT | − | na |
| (PBNJ) | PBNJ120 | CCAACTACC/3SpC3/ | | |
| (SEQ ID NO: 70) | | | | |
| Competitor | G12- | CTACGCCACCAGCTCCA | − | na |
| (PBNJ) | PBNJ100 | ACTA/3SpC3/ | | |
| (SEQ ID NO: 71) | | | | |
| Competitor | G12- | ACGCCACCAGCTCCAAC/ | − | na |
| (PBNJ) | PBNJ80 | 3SpC3/ | | |
| (SEQ ID NO: 72) | | | | |
| Competitor | G12- | CGCCACCAGCTCC/3SpC | − | na |
| (PBNJ) | PBNJ60 | | | |
| (SEQ ID NO: 73) | | | | |
| Competitor | G12- | CCACCAGC/3SpC3/ | − | na |
| (PBNJ) | PBNJ40 | | | |
| Competitor | G12- | ACCAG/3SpC3/ | − | na |
| (PBNJ) | PBNJ20 | | | |
| TaqMan Probe | G12V- | /56FAM/CTACGCCA+AC | − | 2 |
| (SEQ ID NO: 74) | FAM-LNA3 | AGCTCCAAC/3IABkFQ/ | | |
| TaqMan Probe | G12A- | /5HEX/CTACGCCA+GCA | − | 2 |
| (SEQ ID NO: 75) | HEX-LNA4 | GCTCC/3IABkFQ/ | | |
| Competitor | anti-G12D- | CTACGCCA+TCAGCTCC | − | 2 |
| (PBNJ) | PBNJ | AAC/3SpC3/ | | |
| (SEQ ID NO: 76) | | | | |
| Competitor | G12G13_uni- | CTACG+CCA+C+CAGCT | − | 2,3 |
| (PBNJ) | PBNJ_SK2 | CCA/3SpC3/ | | |
| (SEQ ID NO: 77) | | | | |
| TaqMan Probe | G12D- | /56FAM/CTACGCCA+TC | − | 3 |
| (SEQ ID NO: 78) | FAM-LNA3 | AGCTCCAAC/3IABkFQ/ | | |
| TaqMan Probe | G13D- | /5HEX/CCTACG+TCACC | − | 3 |
| (SEQ ID NO: 79) | HEX-LNA1 | AGCTCC/3IABkFQ/ | | |
| Competitor | anti-G12V- | CTACGCCA+ACAGCTCC | − | 3 |
| (PBNJ) | PBNJ-3 | AACT/3SpC3/ | | |
| (SEQ ID NO: 80) | | | | |
| Competitor | anti-G12A- | CTACGCCA+GCAGCTCC | − | 3 |
| (PBNJ) | PBNJ-1 | AAC/3SpC3/ | | |
| (SEQ ID NO: 81) | | | | |

FIG. 11A illustrates the effect of Tₘ on target amplification for KRAS-G12C (top panel) and EGFR-T790M (bottom panel) without PBNJ in a sample having mixed wildtype (WT) and mutant synthetic DNA. Specificity (e.g., on-target amplification) improves with increasing Tₘ, but with the attendant drawback of decreased PCR efficiency (e.g., lower signal output) and decreased signal to noise (STN) ratio. FIG. 11B is an equivalent PCR experiment, but run with PBNJ at 59° C. (annealing/extension). Also varied were PBNJ length (relative to probe length), ranging from 0% (e.g., no PBNJ) to 100% and presence (+)/absence (−) of LNA (at the location of the SNP). PBNJ:probe ratio is 14×. Accordingly, a preferred embodiment to reduce non-specific signal is an at least 80% PBNJ length (relative to probe length) with an LNA, including 100% PBNJ length with an LNA to eliminate at least 95%, including at least 99%, of non-specific signal.

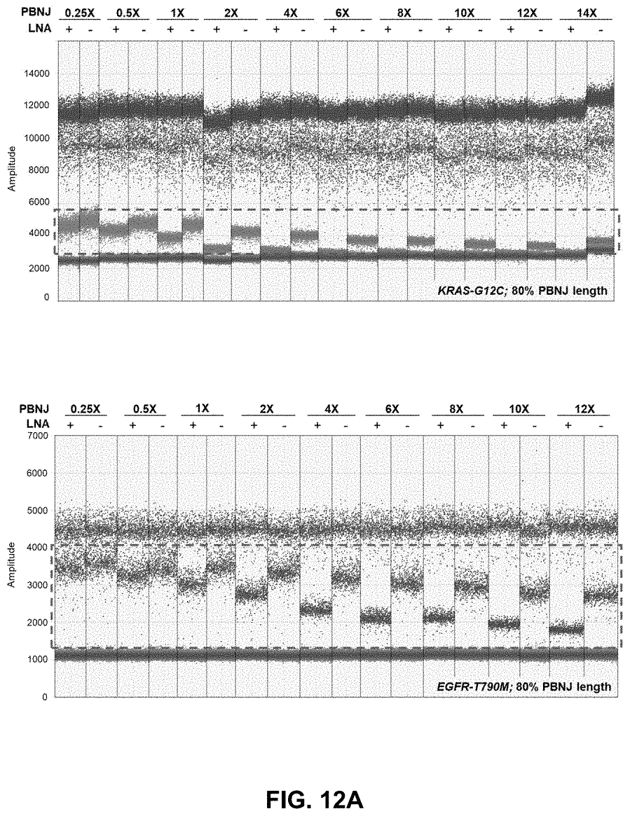

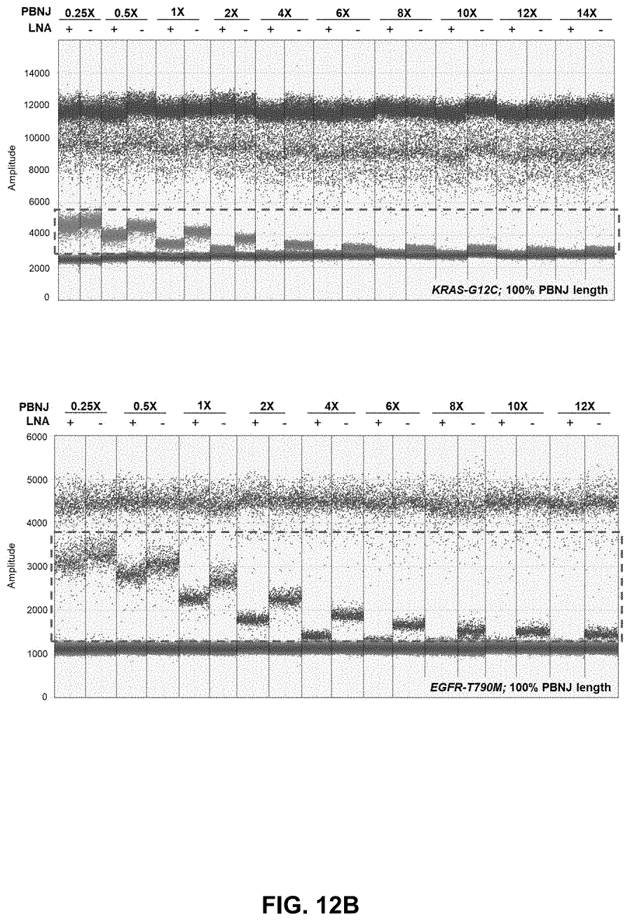

FIG. 12A-12B illustrate effects of various PBNJ ratios (relative to probe), ranging from 0.25× to 14×, presence/absence of LNA, two different PBNJ lengths (80%—FIG. 12A; 100%—FIG. 12B) for a KRAS-G12C target (top panels of each) and EGFR-T790M target (bottom panels of each) on a sample of mixed WT and mutant synthetic DNA. Reduced off-target amplification is demonstrated for increasing PBNJ:probe amount, which may be reflected in terms of concentrations in the PCR assay, and for 100% length PBNJ (relative to probe) for both KRAS and EGFR targets. Accordingly, a preferred embodiment is a 100% length and at least 0.5×PBNJ to 1×PBNJ relative to probe. LNA is preferably present, especially for shorter length PBNJ.

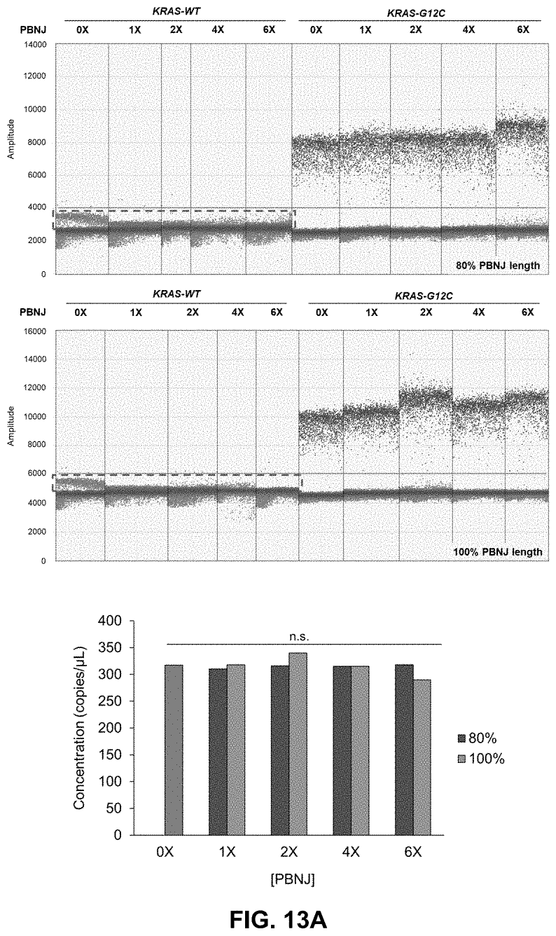

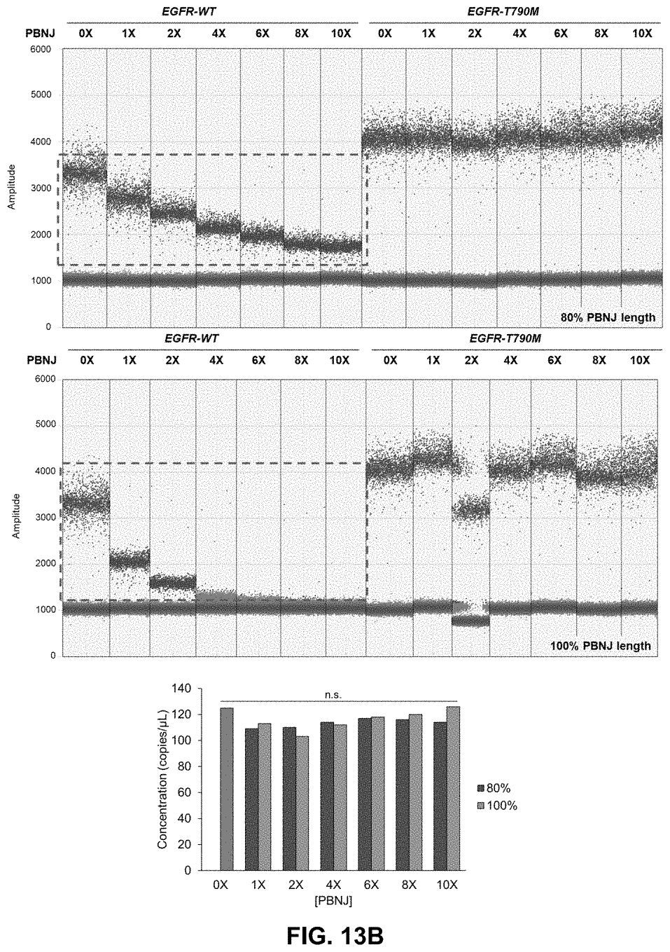

FIGS. 13A-13B confirms that PBNJs do not adversely impact target amplification on pure KRAS-G12C (FIG. 13A) or EGFR-T790M (FIG. 13B) synthetic DNA, for either 80% or 100% PBNJ length (relative to probe). The bottom panels illustrate that any of a PBNJ concentration (ranging from 0× to 6×) does not result in a significantly different concentration of quantified amplicon.

This example demonstrates that the invention is compatible over a range of PCR parameters (melting temperature (Tm), target sequence) and PBNJ-related parameters (LNA+/−; PBNJ to probe length; PBNJ:probe concentration), with the selection of one parameter influencing the selection of another parameter. Although these experiments are based on DNA targets (KRAS and EGFR genes), the methods are certainly compatible with RT-PCR on an RNA target, as described in Example 5.

### Example 5: Use of PBNJs for RT-PCR

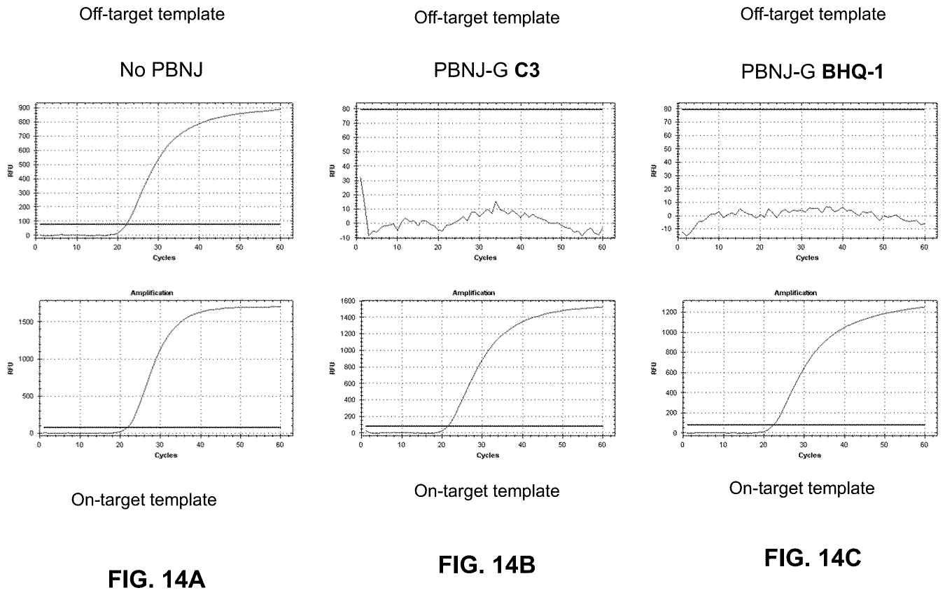

FIG. 14A-14C demonstrate that PBNJs are well-suited for RT-PCR, as reflected by the target that is from SARS-CoV-2 variant, and that there is reduced amplification and attendant detection of non-target sequences when PBNJ is used. This results in on-target amplification that is more reliably detectable, even at very low concentrations relative to reference sequences.

As discussed, the main hurdle for short nucleotide variant assay development is off-target amplification and detection. In many cases, nucleic acid only differs at a single nucleotide. PBNJ blocking technology described herein is particularly suited for this type of application, as demonstrated by PBNJ applied using SARS-CoV-2 variant templates that differ at a single nucleotide within the spike protein gene. Specifically, probes are designed for E484Q mutation, and PBNJs are designed against the E484 wild type sequence (denoted as PBNJ-G). PBNJs are synthesized with either 3′ C3 or BHQ-1 polymerase extension blockers and tested against on- and off-target templates in RT-PCR with TaqPath® (Thermo) on the Bio-Rad CFX-96. As seen in FIGS. 14A-14C, E484 PBNJs are able to efficiently block nonspecific amplification without any loss of on-target detection.

This example demonstrates that PBNJs can be modified with either C3 (and longer chains, e.g., C6 and the like) as well as BHQ-1 and show efficient nonspecific amplification blocking. Furthermore, PBNJs are compatible with RT-PCR.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure.

Every formulation or combination of components described or exemplified herein can be used to practice the invention, unless otherwise stated.

Whenever a range is given in the specification, for example, a temperature range, a ratio range, a time range, or a concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art.

As used herein, “comprising” is synonymous with “including”, “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of discriminating a target sequence from a reference sequence in a biological sample by polymerase chain reaction (PCR), the method comprising the steps of:
providing a labeled probe comprising a fluorophore and a quencher that is complementarity to and specifically binds to the target sequence and may non-specifically bind to the reference sequence;
providing a promiscuity-blocking nucleotide juror oligonucleotide (PBNJ) that specifically binds to the reference sequence and may non-specifically bind to the target sequence, wherein the PBNJ comprises a reference binding region and an extension blocker that prevents elongation by a polymerase;
performing PCR on a solution comprising:
a sample containing the reference and/or target sequence;
the labeled probe;
the PBNJ at a competitive concentration relative to a concentration of the labeled probe;
PCR reagents; and
wherein the PBNJ specific binding to the reference sequence is competitive to the labeled probe binding to the reference sequence and suppresses labeled probe bound to the reference sequence relative to amplification;
thereby discriminating the target sequence from the reference sequence.

2. The method of claim 1, wherein the biological sample is selected from the group consisting of:
viruses, wherein the reference sequence is from a wild-type virus or a parent virus and the target sequence comprises at least one mutation in the reference sequence;
mammalian cells, wherein the reference sequence is reflective of a low-disease condition state and the target sequence has one or more nucleotide changes in the reference sequence reflective of an elevated disease condition risk or the presence of disease;
circulating cell free tumor DNA, wherein the reference sequence is somatic, wild-type sequence and the target sequence originated in a tumor or cancerous cell and has one or more nucleotide changes in the reference sequence reflective of an elevated disease condition risk or the presence of disease;
circulating cell free fetal DNA, wherein the reference sequence is reflective of the maternal DNA sequence and the target sequence has one or more nucleotide changes in the reference sequence reflective of the fetus DNA sequence;
bacteria, wherein the reference sequence is from a wild-type bacterium or one species of bacteria and the target sequence comprises at least one variation in the reference sequence;
fungus, wherein the reference sequence is from a wild-type fungus or one species of fungus and the target sequence comprises at least one variation in the reference sequence; and
plants, wherein the reference sequence is from a wild-type plant or one species of plant and the target sequence comprises at least one variation in the reference sequence.

3. The method of claim 1, wherein the PBNJ eliminates ≥90% detection of non-specific amplification of the reference sequence.

4. The method of claim 1, wherein the reference and target sequences differ by:
a single nucleotide substitution;
a nucleotide insertion of one or more nucleotides; and/or
a nucleotide deletion of one or more nucleotides.

5. The method of claim 1, wherein the reference and target sequences are DNA sequences or RNA sequences.

6. The method of claim 1, wherein the PCR is selected from the group consisting of dPCR, qPCR, RT-dPCR and RT-qPCR.

7. The method of claim 1, wherein the labeled probe is a dual-label probe comprising a fluorescent molecule and at least one quencher molecule.

8. The method of claim 1, wherein the labeled probe is a single-nucleotide variant (SNV)-specific probe having a fluorophore covalently attached to a 5′ end of the probe and a quencher at a 3′ end of the probe or an internal quencher.

9. The method of claim 1, for detection of a SNV, insertion or deletion containing DNA or RNA sequences.

10. The method of claim 1, wherein the PCR is dPCR and the dPCR comprises partition or droplet-based PCR and the PBNJ reduces or eliminates signal associated with a lower efficiency, non-specific off-target amplification, thereby increasing a signal to noise ratio for specific amplification of the target sequence.

11. The method of claim 1, further comprising the steps of:
tuning a probe output amplitude by providing the PBNJ at a lower concentration; and
detecting a plurality of probe output amplitudes for multiplex detection of a plurality of target sequences in a single or a multichannel fluorescence detector.

12. The method of claim 1, wherein the target and reference sequence differ by a single nucleotide mismatch that is a single nucleotide polymorphism (SNP) or is part of a short nucleotide polymorphism.

13. The method of claim 1, wherein the target and reference sequence differ by an insertion.

14. The method of claim 1, wherein the target and reference sequence differ by a deletion.

15. The method of claim 1, wherein the extension blocker is a 3′ carbon-based spacer such as C3, C6, or C12 or a 3′ quencher such as the black hole quencher.

16. The method of claim 1, wherein the PBNJ contains a locked nucleic acid (LNA) at a SNP position.

17. The method of claim 1, wherein the PBNJ has:
a length of between 10 and 50 nucleotides;
a target sequence complementarity to at least a portion of the target sequence that is between 90% and 95%; and
a reference sequence complementarity that is greater than the target sequence complementarity so that:
the binding affinity of the PBNJ to the reference sequence is greater than a binding affinity of the PBNJ to the target sequence;
the binding affinity of the PBNJ to the reference sequence is greater than a binding affinity of the labeled probe to the reference sequence; and/or
the binding affinity of the PBNJ to the target sequence is less than a binding affinity of the labeled probe to the target sequence.

18. The method of claim 1, comprising a plurality of PBNJ's that specifically bind to every possible SNP at a specific location in the target sequence.

19. The method of claim 1, wherein the target sequence is between 10 and 50 nucleotides in length and the probe and the PBNJ are each between 10 and 50 nucleotides in length.

20. The method of claim 1, used in a biological sample to test for mutations associated with an elevated risk or presence of cancer.

21. The method of claim 1, used in a biological sample to test for a variant of a pathogen, including a pathogen that is a virus, a bacteria, or a fungus.

22. The method of claim 1, wherein the biological sample is from wastewater, environmental sample, bodily fluid, tissue, cell culture, or tumor.

23. The method of claim 1, wherein the labeled-probe has a polynucleotide sequence that differs from the PBNJ sequence by one or more nucleotides.

24. The method of claim 1, wherein the PBNJ is a PCR blocker during a PCR amplification cycle to provide enrichment of a target sequence that is part of a mutant sequence.

25. (canceled)

26. A kit for discriminating a target sequence from a reference sequence in a biological sample by polymerase chain reaction (PCR), the kit comprising:
a forward and reverse primer useful for amplifying both reference and target strands;
a labeled probe comprising a fluorophore and a quencher that specifically binds to the target sequence and may non-specifically bind to the reference sequence;
a promiscuity-blocking nucleotide juror oligonucleotide (PBNJ) that specifically binds to the reference sequence and may non-specifically bind to the target sequence;
optionally, a positive control for the reference sequence; and
optionally, a positive control for the target sequence.

27. The kit of claim 26, wherein:
a forward primer is provided at a concentration of between 50 nM and 1100 nM;
a reverse primer is provided at a concentration of between 50 nM and 1100 nM;
the labeled-probe is provided at a concentration of 20-800 nM; and/or
the PBNJ is provided at a concentration that is between 0.25× and 16× the concentration of the labeled-probe.

28. The kit of claim 26, further comprising reagents for dPCR, qPCR RT-dPCR or RT-qPCR.

29. The kit of claim 26, wherein assay reagents are provided for a first reaction not comprising a PBNJ and a second reaction comprising at least one PBNJ.

30. The kit of claim 26, wherein a PBNJ is provided to the wild-type reference sequence of a SARS-CoV-2 mutation selected from the group consisting of Spike residues HV69-70, R408, K417, L452, T478, N501, N679, L704, Q954, and L981.

31. The kit of claim 26, wherein the reference sequence is:
a parental SARS-CoV-2 and the target sequence comprises a variant of SARS-CoV-2 selected from the group consisting of the Alpha variant, Beta variant, Gamma variant, Delta variant, Delta Plus variant, Mu variant, Lambda variant, Omicron variant, and Omicron subvariants; or
a proto-oncogene and the target sequence has a mutation that converts the proto-oncogene to an oncogene indicative of a higher risk of developing cancer or presence of cancer.

32. The kit of claim 31, wherein the reference sequence is a proto-oncogene, including the proto-oncogene that is KRAS with a mutation selected from G12C, G2A, G12D, G12R, or G13D.

33. (canceled)

34. (canceled)

35. The kit of claim 26, wherein the PBNJ is provided at a concentration so that one or more non-specific amplification population is optically indistinguishable from a negative partition population.

36. The kit of claim 26, wherein the PBNJ is provided at a concentration so that a threshold cut-off value for detection of the target sequence is reduced relative to the threshold cut-off value for a method or kit that does not have the PBNJ.

37. The kit of claim 26, wherein the PBNJ has a length of between 80% to 100% of the labeled probe.


SEQUENCE LISTING (81 sequences)

SEQ ID NO: 1 - DNA, 28 bp, synthetic construct
  source 1..28: mol_type = other DNA
tgcgttatagcttggaattctaacaatc

SEQ ID NO: 2 - DNA, 19 bp, synthetic construct
  source 1..19: mol_type = other DNA
ttgctggtgcatgtagaag

SEQ ID NO: 3 - DNA, 26 bp, synthetic construct
  source 1..26: mol_type = other DNA
tgtaattagaggtgatgaagtcagac

SEQ ID NO: 4 - DNA, 19 bp, synthetic construct
  source 1..19: mol_type = other DNA
caagctataacgcagcctg

SEQ ID NO: 5 - DNA, 21 bp, synthetic construct
  source 1..21: mol_type = other DNA
cgtggtgtttattaccctgac

SEQ ID NO: 6 - DNA, 24 bp, synthetic construct
  source 1..24: mol_type = other DNA
caccatcattaaatggtaggacag

SEQ ID NO: 7 - DNA, 21 bp, synthetic construct
  source 1..21: mol_type = other DNA
caaactggaacgattgctgat

SEQ ID NO: 8 - DNA, 22 bp, synthetic construct
  source 1..22: mol_type = other DNA
ccagggcaaactggaaatattg

SEQ ID NO: 9 - DNA, 23 bp, synthetic construct
  source 1..23: mol_type = other DNA
tttccaacccacttatggtgttg

SEQ ID NO: 10 - DNA, 20 bp, synthetic construct
  source 1..20: mol_type = other DNA
tggttccatgctatctctgg

SEQ ID NO: 11 - DNA, 24 bp, synthetic construct
  source 1..24: mol_type = other DNA
cacaccttgtaatggtgttaaagg

SEQ ID NO: 12 - DNA, 20 bp, synthetic construct
  source 1..20: mol_type = other DNA
gaccccaaaatcagcgaaat

SEQ ID NO: 13 - DNA, 24 bp, synthetic construct
  source 1..24: mol_type = other DNA
tctggttactgccagttgaatctg

SEQ ID NO: 14 - DNA, 24 bp, synthetic construct
  source 1..24: mol_type = other DNA
accccgcattacgtttggtggacc

SEQ ID NO: 15 - DNA, 21 bp, synthetic construct
  source 1..21: mol_type = other DNA
acttgtgcccttttggtgaag

SEQ ID NO: 16 - DNA, 26 bp, synthetic construct
  source 1..26: mol_type = other DNA
taggacagaataatcagcaacacagt

SEQ ID NO: 17 - DNA, 16 bp, synthetic construct
  source 1..16: mol_type = other DNA
acgccaccaaatttgc

SEQ ID NO: 18 - DNA, 27 bp, synthetic construct
  source 1..27: mol_type = other DNA
tgttgaaggttttaattgttactctcc

SEQ ID NO: 19 - DNA, 16 bp, synthetic construct
  source 1..16: mol_type = other DNA
acgccaccagatttgc

SEQ ID NO: 20 - DNA, 22 bp, synthetic construct
  source 1..22: mol_type = other DNA
ccagggcaaactggaaagattg

SEQ ID NO: 21 - DNA, 24 bp, synthetic construct
  source 1..24: mol_type = other DNA
caacaccattacaaggtgtgctac

SEQ ID NO: 22 - DNA, 24 bp, synthetic construct
  source 1..24: mol_type = other DNA
caacaccattacaaggtttgctac

SEQ ID NO: 23 - DNA, 29 bp, synthetic construct
  source 1..29: mol_type = other DNA
aattaccggtatagattgtttaggaagtc

SEQ ID NO: 24 - DNA, 22 bp, synthetic construct
  source 1..22: mol_type = other DNA
acaccattacaaggtttgctac

SEQ ID NO: 25 - DNA, 71 bp, synthetic construct
  source 1..71: mol_type = other DNA
atataaacttgtggtagttggagctggtggcgtaggcaagagtgccttgacgatacagctaattcagaatc

SEQ ID NO: 26 - DNA, 71 bp, synthetic construct
  source 1..71: mol_type = other DNA
atataaacttgtggtagttggagcttgtggcgtaggcaagagtgccttgacgatacagctaattcagaatc

SEQ ID NO: 27 - DNA, 71 bp, synthetic construct
  source 1..71: mol_type = other DNA
atataaacttgtggtagttggagctcgtggcgtaggcaagagtgccttgacgatacagctaattcagaatc

SEQ ID NO: 28 - DNA, 22 bp, synthetic construct
  source 1..22: mol_type = other DNA
cctacgccacaagctccaacta

SEQ ID NO: 29 - DNA, 22 bp, synthetic construct
  source 1..22: mol_type = other DNA
cctacgccacgagctccaacta

SEQ ID NO: 30 - DNA, 22 bp, synthetic construct
  source 1..22: mol_type = other DNA
taaggcctgctgaaaatgactg

SEQ ID NO: 31 - DNA, 21 bp, synthetic construct
  source 1..21: mol_type = other DNA
ggtcctgcaccagtaatatgc

SEQ ID NO: 32 - DNA, 16 bp, synthetic construct
  source 1..16: mol_type = other DNA
gcctgctgggcatctg

SEQ ID NO: 33 - DNA, 23 bp, synthetic construct
  source 1..23: mol_type = other DNA
tctttgtgttcccggacatagtc

SEQ ID NO: 34 - DNA, 21 bp, synthetic construct
  source 1..21: mol_type = other DNA
cagctcatcatgcagctcatg

SEQ ID NO: 35 - DNA, 25 bp, synthetic construct
  source 1..25: mol_type = other DNA
tgcagctcatcacgcagctcatgcc

SEQ ID NO: 36 - DNA, 21 bp, synthetic construct
  source 1..21: mol_type = other DNA
cagctcatcacgcagctcatg

SEQ ID NO: 37 - DNA, 17 bp, synthetic construct
  source 1..17: mol_type = other DNA
agctcatcacgcagctc

SEQ ID NO: 38 - DNA, 13 bp, synthetic construct
  source 1..13: mol_type = other DNA
tcatcacgcagct

SEQ ID NO: 39 - DNA, 25 bp, synthetic construct
  source 1..25: mol_type = other DNA
tgcagctcatcacgcagctcatgcc

SEQ ID NO: 40 - DNA, 21 bp, synthetic construct
  source 1..21: mol_type = other DNA
cagctcatcacgcagctcatg

SEQ ID NO: 41 - DNA, 17 bp, synthetic construct
  source 1..17: mol_type = other DNA
agctcatcacgcagctc

SEQ ID NO: 42 - DNA, 13 bp, synthetic construct
  source 1..13: mol_type = other DNA
tcatcacgcagct

SEQ ID NO: 43 - DNA, 15 bp, synthetic construct
  source 1..15: mol_type = other DNA
cttcggctgcctcct

SEQ ID NO: 44 - DNA, 16 bp, synthetic construct
  source 1..16: mol_type = other DNA
cttcggctccctcctg

SEQ ID NO: 45 - DNA, 16 bp, synthetic construct
  source 1..16: mol_type = other DNA
cttcggcagcctcctg

SEQ ID NO: 46 - DNA, 18 bp, synthetic construct
  source 1..18: mol_type = other DNA
ctgacgtgcctctccctc

SEQ ID NO: 47 - DNA, 17 bp, synthetic construct
  source 1..17: mol_type = other DNA
gtgaggcagatgcccag

SEQ ID NO: 48 - DNA, 18 bp, synthetic construct
  source 1..18: mol_type = other DNA
tgtccacgctggccatca

SEQ ID NO: 49 - DNA, 18 bp, synthetic construct
  source 1..18: mol_type = other DNA
tgtccacgatggccatca

SEQ ID NO: 50 - DNA, 19 bp, synthetic construct
  source 1..19: mol_type = other DNA
tggagaagctcccaaccaa

SEQ ID NO: 51 - DNA, 20 bp, synthetic construct
  source 1..20: mol_type = other DNA
cttatacaccgtgccgaacg

SEQ ID NO: 52 - DNA, 15 bp, synthetic construct
  source 1..15: mol_type = other DNA
cggagcccagcactt

SEQ ID NO: 53 - DNA, 15 bp, synthetic construct
  source 1..15: mol_type = other DNA
cggagcccagcactt

SEQ ID NO: 54 - DNA, 17 bp, synthetic construct
  source 1..17: mol_type = other DNA
tccggagctcagcactt

SEQ ID NO: 55 - DNA, 17 bp, synthetic construct
  source 1..17: mol_type = other DNA
tccggagcacagcactt

SEQ ID NO: 56 - DNA, 15 bp, synthetic construct
  source 1..15: mol_type = other DNA
cggaggccagcactt

SEQ ID NO: 57 - DNA, 23 bp, synthetic construct
  source 1..23: mol_type = other DNA
gcagcatgtcaagatcacagatt

SEQ ID NO: 58 - DNA, 25 bp, synthetic construct
  source 1..25: mol_type = other DNA
cctccttctgcatggtattctttct

SEQ ID NO: 59 - DNA, 17 bp, synthetic construct
  source 1..17: mol_type = other DNA
cagtttggccagcccaa

SEQ ID NO: 60 - DNA, 15 bp, synthetic construct
  source 1..15: mol_type = other DNA
tttggcccgcccaaa

SEQ ID NO: 61 - DNA, 16 bp, synthetic construct
  source 1..16: mol_type = other DNA
acccagcagtttggcc

SEQ ID NO: 62 - DNA, 16 bp, synthetic construct
  source 1..16: mol_type = other DNA
acccagctgtttggcc

SEQ ID NO: 63 - DNA, 20 bp, synthetic construct
  source 1..20: mol_type = other DNA
aggcctgctgaaaatgactg

SEQ ID NO: 64 - DNA, 20 bp, synthetic construct
  source 1..20: mol_type = other DNA
gctgtatcgtcaaggcactc

SEQ ID NO: 65 - DNA, 21 bp, synthetic construct
  source 1..21: mol_type = other DNA
ctacgccacaagctccaacta

SEQ ID NO: 66 - DNA, 26 bp, synthetic construct
  source 1..26: mol_type = other DNA
tgcctacgccaccagctccaactacc

SEQ ID NO: 67 - DNA, 21 bp, synthetic construct
  source 1..21: mol_type = other DNA
ctacgccaccagctccaacta

SEQ ID NO: 68 - DNA, 17 bp, synthetic construct
  source 1..17: mol_type = other DNA
acgccaccagctccaac

SEQ ID NO: 69 - DNA, 13 bp, synthetic construct
  source 1..13: mol_type = other DNA
cgccaccagctcc

SEQ ID NO: 70 - DNA, 26 bp, synthetic construct
  source 1..26: mol_type = other DNA
tgcctacgccaccagctccaactacc

SEQ ID NO: 71 - DNA, 21 bp, synthetic construct
  source 1..21: mol_type = other DNA
ctacgccaccagctccaacta

SEQ ID NO: 72 - DNA, 17 bp, synthetic construct
  source 1..17: mol_type = other DNA
acgccaccagctccaac

SEQ ID NO: 73 - DNA, 13 bp, synthetic construct
  source 1..13: mol_type = other DNA
cgccaccagctcc

SEQ ID NO: 74 - DNA, 19 bp, synthetic construct
  source 1..19: mol_type = other DNA
ctacgccaacagctccaac

SEQ ID NO: 75 - DNA, 16 bp, synthetic construct
  source 1..16: mol_type = other DNA
ctacgccagcagctcc

SEQ ID NO: 76 - DNA, 19 bp, synthetic construct
  source 1..19: mol_type = other DNA
ctacgccatcagctccaac

SEQ ID NO: 77 - DNA, 17 bp, synthetic construct
  source 1..17: mol_type = other DNA
ctacgccaccagctcca

SEQ ID NO: 78 - DNA, 19 bp, synthetic construct
  source 1..19: mol_type = other DNA
ctacgccatcagctccaac

SEQ ID NO: 79 - DNA, 17 bp, synthetic construct
  source 1..17: mol_type = other DNA
cctacgtcaccagctcc

SEQ ID NO: 80 - DNA, 20 bp, synthetic construct
  source 1..20: mol_type = other DNA
ctacgccaacagctccaact

SEQ ID NO: 81 - DNA, 19 bp, synthetic construct
  source 1..19: mol_type = other DNA
ctacgccagcagctccaac